(19) United States
(12) Patent Application Publication
(10) Pub. No.: US 2012/0141539 A1
(43) Pub. Date: Jun. 7, 2012

(54) METHODS FOR MODULATING TOLL-LIKE RECEPTOR MEDIATED ACTIVATION OF CELLS OF THE INNATE SYSTEM BY MODULATING XBP-1 ACTIVITY

(75) Inventor: Laurie H Glimcher, West Newton, MA (US)

(73) Assignee: PRESIDENT AND FELLOWS OF HARVARD COLLEGE, Cambridge, MA (US)

(21) Appl. No.: 13/375,718

(22) PCT Filed: Jun. 2, 2010
(86) PCT No.: PCT/US10/37113

§ 371 Date: Feb. 23, 2012

Related U.S. Application Data
(60) Provisional application No. 61/183,360, filed on Jun. 2, 2009.

(51) Int. Cl. A61K 31/7088; A61P 37/04; C12N 5/0786; C12Q 1/68; C40B 30/00; C40B 30/06

(52) U.S. Cl. 424/278.1; 506/7; 506/10; 435/375; 435/6.1

(57) ABSTRACT

The invention provides methods and compositions for modulating the activity of XBP-I protein, or a protein in a signal transduction pathway involving XBP-I to modulate the TLR-mediated activation of cells of the innate immune system. Enhancing the TLR-mediated activation of cells of the innate immune system enhance inflammatory responses. The present invention also pertains to methods for identifying compounds that modulate Toll-like receptor-mediated signaling.

### Government Funding

Work described herein was supported, at least in part, under grants AI32412 and AI56296 awarded by the National Institutes of Health. The U.S. government, therefore, will have certain rights in this invention.

### BACKGROUND OF THE INVENTION

New methods of treating infection have become increasingly important in the age of multiple antibiotic resistance. The transcription factor XBP-1 was identified as a key regulator of the mammalian unfolded protein response (UPR) or endoplasmic reticulum (ER) stress response, which is activated by environmental stressors such as protein overload that require increased ER capacity (D. Ron, P. Walter (2007) Nat Rev Mol Cell Biol 8, 519). XBP-1 is activated by a post-transcriptional modification of its mRNA by IRE-1 alpha, an ER localizing proximal sensor of ER stress (M. Calfon et al. (2002) Nature 415, 92; H. Yoshida, et al. (2001) Cell 107, 881; X. Shen et al. (2001) Cell 107, 893). Upon ER stress, IRE-1 alpha induces an unconventional splicing of XBP-1 mRNA by using its endoribonuclease activity to generate a mature mRNA encoding an active transcription factor, XBP-1s, which directly binds to the promoter region of ER chaperone genes to promote transcription (A. L. Shaffer et al. (2004) Immunity 21, 81; A. H. Lee, et al. (2003) Mol Cell Biol 23, 7448; D. Acosta-Alvear et al. (2007) Mol Cell 27, 53). Mice deficient in XBP-1 display severe abnormalities in the development and function of professional secretory cells, such as plasma B cells and pancreatic acinar cells (N. N. Iwakoshi et al. (2003) Nat Immunol 4, 321; A. H. Lee, et al. (2005) Embo J 24, 4368) and intestinal Paneth cells (Kaser, et al (2008) Cell).

The further identification of novel targets for treating infection would be of great benefit.

### SUMMARY OF THE INVENTION

The present invention demonstrates, inter alia, a role for the transcription factor XBP-1 in infection with organisms that bind to Toll-like receptors (TLRs). As described in the appended Examples, it has been discovered that XBP-1 plays an important role in modulating toll-like receptor (TLR)-mediated responses and that enhancing XBP-1 activity can amplify the innate immune response. Accordingly, among other things, innate immunity can be boosted in the setting of vaccination or natural infection by inducing the IRE1/XBP1 arm of the endoplasmic reticulum (ER) stress response. That is to say that ER stress, properly harnessed, can act as a novel adjuvant in macrophages and dendritic cells. This is a novel function for one arm of the ER stress response which can be used, e.g., to activate innate immunity in response to pathogens

Accordingly, in one aspect, the invention pertains to a method of identifying a compound that is useful in increasing Toll like receptor-(TLR) mediated signaling comprising, a) providing an indicator composition comprising an XBP-1 polypeptide; b) contacting the indicator composition with each member of a library of compounds; c) determining the effect of the compound on XBP-1 activity; d) selecting a compound of interest that increases XBP-1 activity as compared to an appropriate control; e) determining the effect of the compound on TLR-mediated signaling; f) selecting a compound of interest that increases TLR-mediated signaling as compared to an appropriate control, thereby identifying the compound as useful in increasing TLR-mediated signaling.

In another aspect, the invention pertains to a method of identifying a compound that is useful in increasing Toll like receptor-(TLR) mediated signaling comprising, a) providing an indicator composition comprising an IRE-1 polypeptide; b) contacting the indicator composition with each member of a library of compounds; c) determining the effect of the compound on IRE-1 activity; d) selecting a compound of interest that increases IRE-1 activity as compared to an appropriate control; e) determining the effect of the compound on TLR-mediated signaling; f) selecting a compound of interest that increases TLR-mediated signaling as compared to an appropriate control, thereby identifying the compound as useful in increasing TLR-mediated signaling.

In yet another aspect, the invention pertains to a method of identifying a compound that is useful in decreasing Toll like receptor-(TLR) mediated signaling comprising, a) providing an indicator composition comprising an XBP-1 polypeptide; b) contacting the indicator composition with each member of a library of compounds; c) determining the effect of the compound on XBP-1 activity; d) selecting a compound of interest that decreases XBP-1 activity as compared to an appropriate control; and e) determining the effect of the compound on TLR-mediated signaling; f) selecting a compound of interest that decreases TLR-mediated signaling as compared to an appropriate control, thereby identifying the compound as useful in decreasing TLR-mediated signaling.

In still another aspect, the invention pertains to a method of identifying a compound that is useful in decreasing like receptor-(TLR) mediated signaling comprising, a) providing an indicator composition comprising an IRE-1 polypeptide; b) contacting the indicator composition with each member of a library of compounds; c) determining the effect of the compound on IRE-1 activity; d) selecting a compound of interest that decreases IRE-1 activity as compared to an appropriate control; e) determining the effect of the compound on TLR-mediated signaling; f) selecting a compound of interest that decreases TLR-mediated signaling as compared to an appropriate control, thereby identifying the compound as useful in increasing TLR-mediated signaling.

In one embodiment, the effect of the compound on TLR-mediated signaling is determined by measuring the effect of the compound on the production of a proinflammatory cytokine.

In another embodiment, the effect of the compound on the production of a proinflammatory cytokine is is determined by measuring the effect of the compound on sustained production of the proinflammatory cytokine.

In another embodiment, the proinflammatory cytokine is IL-6.

In another embodiment, the proinflammatory cytokine is IFNβ.

In another embodiment, the proinflammatory cytokine is ISG15.

In another embodiment, the activity of XBP-1 is determined by measuring XBP-1 splicing.

In another embodiment, the activity of XBP-1 is determined by assaying XBP-1 protein levels.

In another embodiment, the method further comprises determining the activation of MyD88, TRIF, TRAF6, or NADPH oxidase.

In another embodiment, the activity of IRE-1 is determined by measuring IRE-1 kinase activity.

In another embodiment, the activity of IRE-1 is determined by measuring IRE-1 endoribonuclease activity.

In another embodiment, the activity of IRE-1 is determined by measuring the binding of IRE-1 to XBP-1.

In another embodiment, the activity of IRE-1 is determined by measuring IRE-I protein levels.

In another embodiment, the TLR-mediated signaling is TLR2-mediated signaling.

In another embodiment, the TLR-mediated signaling is TLR4-mediated signaling.

In another embodiment, the TLR-mediated signaling is TLR5-mediated signaling.

In another embodiment, the indicator composition is a hematopoietic cell.

In another embodiment, the cell has been engineered to express the XBP-1 polypeptide by introducing into the cell an expression vector encoding the polypeptide.

In another embodiment, the cell is a macrophage.

In another embodiment, the cell is a dendritic cell.

In another embodiment, the cell is under ER stress.

In another embodiment, the cell is contacted with an agent that activates TLR signaling.

In another embodiment, the indicator composition is a cell free composition.

In another embodiment, the method further comprises determining the effect of the identified compound on PERK and/or ATF6 activity.

In another embodiment, the method further comprises determining the effect of the compound on XBP-1 mRNA splicing and/or XBP-1 protein production.

In another embodiment, the method further comprises determining the effect of the compound on the activation of immunoglobulin production.

In another embodiment, the method further comprises determining the effect of the compound on the activation of LPS-stimulated antibody production.

In another embodiment, the method further comprises determining the effect of the compound on a response of an immune cell to a bacterial pathogen.

In another embodiment, the method further comprises determining the effect of the identified test compound on an innate immune response in a non-human animal, comprising administering the test compound to the animal, measuring an effect of TLR-mediated signaling on an innate immune response in the animal in the presence and absence of the test compound, and selecting a compound that modulates TLR-mediated signaling in the animal to thereby determine the effect of the test compound identified on an innate immune response in the animal.

In one embodiment, the non-human animal model is a model of infection with a bacterial pathogen.

In another aspect, the invention pertains to a method for increasing Toll like receptor signaling in a macrophage, comprising contacting a macrophage with an agent that increases the biological activity of XBP-1 in the macrophage, wherein the agent is selected from the group consisting of: a nucleic acid molecule encoding an XBP-1 polypeptide, a nucleic acid molecule encoding an XBP-1 polypeptide, or combinations thereof, such that Toll like receptor signaling is increased in the macrophage.

In another embodiment, the step of contacting occurs in vitro.

In another embodiment, the step of contacting occurs in vivo.

In another embodiment, TLR-mediated signaling is initiated by an agent that binds to a Toll like receptor selected from the group consisting of Toll like receptor 2, 4, and 5.

In another embodiment, the agent activates MyD88, TRIF, TRAF6, or NADPH oxidase.

In another embodiment, the agent is a pathogen.

In another embodiment, the agent is selected from the group consisting of: lipopolysaccharide (LPS), lipoteichoic acid, PAM3CSK4, or FSL1.

In another embodiment, the method further comprises assaying for increases TLR-mediated signaling.

In another embodiment, the method further comprises stimulating ER stress in the cell.

### DETAILED DESCRIPTION

The instant invention is based, at least in part, on the discovery that XBP-1 plays a role in modulating the activation of cells of the innate immune system. In particular, it has been discovered that the transcription factor XBP-1, best known as a key regulator of the Unfolded Protein Response (UPR), plays a key role in modulating TLR-mediated responses in macrophages and dendritic cells. As described herein, it has been shown that TLR4 and TLR2 initiate a biased endoplasmic reticulum (ER)-stress response with selective activation of the inositol requiring enzyme 1 (IRE1) and its downstream target, the X box binding protein 1 (XBP-1) transcription factor, but repress other ER-stress pathways. Previously described XBP-1 ER stress target genes are not induced by TLR signaling. Instead, TLR-activated XBP-1 is required for optimal sustained production of proinflammatory cytokines in macrophages. XBP-1 deficiency markedly increases bacterial burden in animals infected with the TLR2-activating human pathogen Francisella tularensis.

Certain terms are first defined so that the invention may be more readily understood.

### I. Definitions

The “innate immune system” comprises the cells and mechanisms that defend the host from infection by other organisms, in a non-specific manner. The innate system, unlike the adaptive immune system, does not confer long-lasting or protective immunity to a host, e.g., antibody protection, but rather provides immediate defense against infection. The innate system is an evolutionarily older defense strategy, and is the dominant immune system found in plants, fungi, insects, and in primitive multicellular organisms, and in all classes of plant and animal life.

The major functions of the vertebrate innate immune system include recruiting immune cells to sites of infection and inflammation, through the production of cytokines; activation of the complement cascade to identify bacteria, activate cells and to promote clearance of dead cells or antibody complexes; identification and removal of foreign substances present in organs, tissues, the blood and lymph; and activation of the adaptive immune system through antigen presentation.

The innate immune system recognizes key molecular signatures of pathogens or “pathogen associated molecular patterns” (PAMPs), also referred to as “microbe-associated molecular patterns” (MAMPs) (R. Medzhitov, Nature 449, 819 (Oct. 18, 2007)) that include carbohydrates (e.g. structural components, e.g. lipopolysaccharide or LPS, mannose, peptidoglycans (PGN)), nucleic acids (e.g. bacterial or viral DNA or RNA, dsRNA, DNA), peptidoglycans and lipotechoic acids (from Gram positive bacteria), N-formylmethionine, lipoproteins and fungal glucans. The host organism harbors a group of receptors referred to as “pathogen recognition receptors” (“PRRs”) that recognize these PAMPs, the best studied of which is the “Toll like receptors” (“TLRs”) (K. J. Ishii, S. Koyama, A. Nakagawa, C. Coban, S. Akira, Cell Host Microbe 3, 352 (Jun. 12, 2008)).

Pathogen recognition receptors, also referred to as “primitive pattern recognition receptors” are proteins expressed by cells of the immune system to identify molecules associated with microbial pathogens or cellular stress. PRRs are classified according to their ligand specificity, function, localization and/or evolutionary relationships. On the basis of function, PRRs may be divided into endocytic PRRs or signaling PRRs. Signaling PRRs include the large families of membrane-bound Toll-like receptors and cytoplasmic NOD-like receptors. Endocytic PRRs promote the attachment, engulfment and destruction of microorganisms by phagocytes, without relaying an intracellular signal. Endocytic PRRs recognize carbohydrates and include mannose receptors of macrophages, glucan receptors present on all phagocytes and scavenger receptors that recognize charged ligands, are found on all phagocytes and mediate removal of apoptotic cells.

TLRs are single membrane-spanning non-catalytic receptors that recognize structurally conserved molecules derived from microbes, e.g., PAMPs. TLRs together with the Interleukin-1 receptor, e.g., IL-1 and IL-18) form a receptor superfamily, known as the “Interleukin-1 Receptor/Toll-Like Receptor Superfamily”; members of this family are characterized structurally by an extracellular leucine-rich repeat (LRR) domain, a conserved pattern of juxtamembrane cysteine residues, and an intracytoplasmic signaling domain (Toll/IL-1 resistance or Toll-IL-1 receptor (TIR)) domain that forms a platform for downstream signaling by recruiting (via TIR-TIR interactions) TIR domain-containing adapters including MyD88, TIR domain-containing adaptor (TIRAP), and TIR domain-containing adaptor inducing IFNβ (TRIF) (L. A. O'Neill, A. G. Bowie, Nat Rev Immunol 7, 353 (May 1, 2007)).

There are three subgroups of TIR domains. Proteins with subgroup 1 TIR domains are receptors for interleukins that are produced by macrophages, monocytes and dendritic cells and all have extracellular Immunoglobulin (Ig) domains. Proteins with subgroup 2 TIR domains are classical TLRs, and bind directly or indirectly to molecules of microbial origin, e.g., TLR5, TLR4 and TLR2. A third subgroup of proteins containing TIR domains consists of adaptor proteins that are exclusively cytosolic and mediate signaling from proteins of subgroups 1 and 2.

The nucleotide and amino acid sequences of TLRs are known and can be found at, for example, GenBank Accession Nos. gi:41350336, gi:13507602 (TLR1 human and mouse, respectively); gi:68160956, gi:158749637, gi:42476288 (TLR2 human, mouse, and rat, respectively); gi:19718735, GI:146149239, GI:38454315 (TLR3 human, mouse, and rat, respectively); GI:88758616, GI:118130391, gi:25742798 (TLR4 human, mouse, and rat, respectively); gi:124248535, gi:124248589, gi:109498326 (TLR5 human, mouse, and rat, respectively); gi:20143970, gi:157057100, gi:46485392 (TLR6 human, mouse, and rat, respectively); gi:67944638, gi:141803199, gi:147900683 (TLR7 human, mouse, and rat, respectively); gi:156071526, gi:126723494 (TLR8 human and mouse, respectively); gi:20302169, gi:157057165 (TLR9 human and mouse, respectively); there are two isoforms of TLR 10 in human, gi:62865620 and gi:62865617, gi:109499688 (TLR10 rat); gi:148539899, gi:221307462 (TLR11 mouse and rat, respectively); gi:148539900 (TLR12 mouse); gi:45429998 (TLR13 mouse).

TLR-mediated signaling in response to PAMPs is a sequential cascade of transcriptional regulatory events that vary depending on the TLR agonists, cell types involved and pathogenicity of the microbe. Individual genes (notably proinflammatory cytokines, e.g., IL-1 (alpha and beta), IL-6, IL-18, TNF-α) are induced transiently and then repressed reflecting the ability that the innate immune system has to interpret the infection and orchestrate appropriate responses while promoting resolution (T. Ravasi, C. A. Wells, D. A. Hume, Bioessays 29, 1215 (Nov. 15, 2007); J. C. Roach et al., Proc Natl Acad Sci USA 104, 16245 (Oct. 9, 2007); M. Gilchrist et al., Nature 441, 173 (May 11, 2006)). Nuclear factor-kappaB (NF-κB), the best characterized transcription factor downstream of TLRs, is activated by virtually all TLRs, e.g., TLR5, TLR4 and TLR2, through MyD88 or TRIF dependent pathways and is crucial for the production of proinflammatory cytokines. However, bacterial products are not the only signals that modulate innate immune responses-signals produced by stressed or damaged tissues have also been suggested to modulate the inflammatory response (H. Kono, K. L. Rock, Nat Rev Immunol 8, 279 (Apr. 1, 2008); R. Medzhitov, Nature 454, 428 (Jul. 24, 2008)).

The nucleotide and amino acid sequences of MyD88 are known and can be found at, for example, GenBank Accession Nos. gi:197276653, gi:31543276, gi:37693502 (human, mouse, rat, respectively).

The nucleotide and amino acid sequences of TIR domain-containing adaptor (TIRAP) are known and can be found at, for example, GenBank Accession Nos. gi:89111121 and gi:89111123 (two isoforms in himan), gi:16905130 and gi:109483246 (mouse and rat).

The nucleotide and amino acid sequences of TIR domain-containing adaptor inducing IFNβ (TRIF) are known and can be found at, for example, GenBank Accession Nos. gi:197209874 and gi:144227224 (human and mouse, respctcively).

As used herein, the term “XBP-1” refers to the X-box binding protein. XBP-1 is a basic region leucine zipper (b-zip) transcription factor isolated independently by its ability to bind to a cyclic AMP response element (CRE)-like sequence in the mouse class II MHC Aα gene or the CRE-like site in the HTLV-1 21 base pair enhancer, and subsequently shown to regulate transcription of both the DRα and HTLV-1 ltr gene.

Like other members of the b-zip family, XBP-1 has a basic region that mediates DNA-binding and an adjacent leucine zipper structure that mediates protein dimerization. Deletional and mutational analysis has identified transactivation domains in the C-terminus of XBP-1 in regions rich in acidic residues, glutamine, serine/threonine and proline/glutamine. XBP-1 is present at high levels in plasma cells in joint synovium in patients with rheumatoid arthritis. In human multiple myeloma cells, XBP-1 is selectively induced by IL-6 treatment and implicated in the proliferation of malignant plasma cells. XBP-1 has also been shown to be a key factor in the transcriptional regulation of molecular chaperones and to enhance the compensatory UPR (Calfon et al., Nature 415, 92 (2002); Shen et al., Cell 107:893 (2001); Yoshida et al., Cell 107:881 (2001); Lee et al., Mol. Cell Biol. 23:7448 (2003); each of which is incorporated herein by reference).

The amino acid sequence of XBP-1 is described in, for example, Liou, H-C. et. al. (1990) Science 247:1581-1584 and Yoshimura, T. et al. (1990) EMBO J. 9:2537-2542. The amino acid sequence of mammalian homologs of XBP-1 are described in, for example, in Kishimoto T. et al., (1996) Biochem. Biophys. Res, Commun, 223:746-751 (rat homologue). Exemplary proteins intended to be encompassed by the term “XBP-1” include those having amino acid sequences disclosed in GenBank with accession numbers A36299 [gi:105867]; AF443192 [gi: 18139942] (spliced murine XBP-1); P17861 [gi:139787]; CAA39149 [gi:287645]; AF027963 [gi: 13752783] (murine unspliced XBP-1); BAB82982.1 [gi:18148382] (spliced human XBP-1); BAB82981 [gi:18148380] (human unspliced XBP-1); and BAA82600 [gi:5596360] or e.g., encoded by nucleic acid molecules such as those disclosed in GenBank with accession numbers AF027963 [gi: 13752783]; NM_{—}013842 [gi:13775155] (spliced murine XBP-1); or M31627 [gi:184485] (unspliced murine XBP-1); AB076384 [gi:18148381] (spliced human XBP-1); or AB076383 [gi:18148379] (human unspliced XBP-1); gi:51948392 (rat). XBP-1 is also referred to in the art as TREB5 or HTF (Yoshimura et al. 1990. EMBO Journal. 9:2537; Matsuzaki et al. 1995. J. Biochem. 117:303).

There are two forms of XBP-1 protein, unspliced and spliced, which differ markedly in their sequence and activity. Unless the form is referred to explicitly herein, the term “XBP-1” as used herein includes both the spliced and unspliced forms.

As used herein, the term “spliced XBP-1” or “XBP-1s” refers to the spliced, processed form of the mammalian XBP-1 mRNA or the corresponding protein. Human and murine XBP-1 mRNA contain an open reading frame (ORF1) encoding bZIP proteins of 261 and 267 amino acids, respectively. Both mRNAs also contain another ORF, ORF2, partially overlapping but not in frame with ORF1. ORF2 encodes 222 amino acids in both human and murine cells. Human and murine ORF1 and ORF2 in the XBP-1 mRNA share 75% and 89% identity respectively. In response to ER stress, XBP-1 mRNA is processed by the ER transmembrane endoribonuclease and kinase IRE-1 which excises an intron from XBP-1 mRNA. In murine and human cells, a 26 nucleotide intron is excised. The boundaries of the excised introns are encompassed in an RNA structure that includes two loops of seven residues held in place by short stems. The RNA sequences 5′ to 3′ to the boundaries of the excised introns form extensive base-pair interactions. Splicing out of 26 nucleotides in murine and human cells results in a frame shift at amino acid 165 (the numbering of XBP-1 amino acids herein is based on GenBank accession number NM_{—}013842 [gi:13775155] (spliced murine XBP-1) and one of ordinary skill in the art can determine corresponding amino acid numbers for XBP-1 from other organisms, e.g., by performing a simple alignment). This causes removal of the C-terminal 97 amino acids from the first open reading frame (ORF1) and addition of the 212 amino from ORF2 to the N-terminal 164 amino acids of ORF1 containing the b-ZIP domain. In mammalian cells, this splicing event results in the conversion of a 267 amino acid unspliced XBP-1 protein to a 371 amino acid spliced XBP-1 protein. The spliced XBP-1 then translocates into the nucleus where it binds to its target sequences to induce their transcription.

As used herein, the term “unspliced XBP-1” refers to the unprocessed XBP-1 mRNA or the corresponding protein. As set forth above, unspliced murine XBP-1 is 267 amino acids in length and spliced murine XBP-1 is 371 amino acids in length. The sequence of unspliced XBP-1 is known in the art and can be found, e.g., Liou, H-C. et. al. (1990) Science 247:1581-1584 and Yoshimura, T. et al. (1990) EMBO J. 9:2537-2542, or at GenBank accession numbers: AF443192 [gi: 18139942] (amino acid spliced murine XBP-1); AF027963 [gi: 13752783] (amino acid murine unspliced XBP-1); NM_{—}013842 [gi:13775155] (nucleic acid spliced murine XBP-1); or M31627 [gi:184485] (nucleic acid unspliced murine XBP-1.

As used herein, the term “ratio of spliced to unspliced XBP-1” refers to the amount of spliced XBP-1 present in a cell or a cell-free system, relative to the amount or of unspliced XBP-1 present in the cell or cell-free system. “The ratio of unspliced to spliced XBP-1” refers to the amount of unspliced XBP-1 compared to the amount of unspliced XBP-1. “Increasing the ratio of spliced XBP-1 to unspliced XBP-1” encompasses increasing the amount of spliced XBP-1 or decreasing the amount of unspliced XBP-1 by, for example, promoting the degradation of unspliced XBP-1. Increasing the ratio of unspliced XBP-1 to spliced XBP-1 can be accomplished, e.g., by decreasing the amount of spliced XBP-1 or by increasing the amount of unspliced XBP-1. Levels of spliced and unspliced XBP-1 an be determined as described herein, e.g., by comparing amounts of each of the proteins which can be distinguished on the basis of their molecular weights or on the basis of their ability to be recognized by an antibody. In another embodiment described in more detail below, PCR can be performed employing primers with span the splice junction to identify unspliced XBP-1 and spliced XBP-1 and the ratio of these levels can be readily calculated.

As used herein, the term “Unfolded Protein Response” (UPR) or the “Unfolded Protein Response pathway” refers to an adaptive response to the accumulation of unfolded proteins in the ER and includes the transcriptional activation of genes encoding chaperones and folding catalysts and protein degrading complexes as well as translational attenuation to limit further accumulation of unfolded proteins. Both surface and secreted proteins are synthesized in the endoplasmic reticulum (ER) where they need to fold and assemble prior to being transported.

Since the ER and the nucleus are located in separate compartments of the cell, the unfolded protein signal must be sensed in the lumen of the ER and transferred across the ER membrane and be received by the transcription machinery in the nucleus. The unfolded protein response (UPR) performs this function for the cell. Activation of the UPR can be caused by treatment of cells with reducing agents like DTT, by inhibitors of core glycosylation like tunicamycin or by Ca-ionophores that deplete the ER calcium stores. First discovered in yeast, the UPR has now been described in C. elegans as well as in mammalian cells. In mammals, the UPR signal cascade is mediated by three types of ER transmembrane proteins: the protein-kinase and site—specific endoribonuclease IRE-1; the eukaryotic translation initiation factor 2 kinase, PERK/PEK; and the transcriptional activator ATF6. If the UPR cannot adapt to the presence of unfolded proteins in the ER, an apoptotic response is initiated leading to the activation of JNK protein kinase and caspases 7, 12, and 3. The most proximal signal from the lumen of the ER is received by a transmembrane endoribonuclease and kinase called IRE-1. Following ER stress, IRE-1 initiates splicing of the XBP-1 mRNA, the spliced version of which, activates the UPR.

Eukaryotic cells respond to the presence of unfolded proteins by upregulating the transcription of genes encoding ER resident protein chaperones such as the glucose-regulated BiP/Grp74, GrP94 and CHOP genes, folding catalysts and protein degrading complexes that assist in protein folding.

As used herein, the term “modulation of the UPR” includes both upregulation and downregulation of the UPR. As used herein the term “UPRE” refers to UPR elements upstream of certain genes which are involved in the activation of these genes in response, e.g., to signals sent upon the accumulation of unfolded proteins in the lumen of the endoplasmic reticulum, e.g., EDEM, Herp, e.g., ER stress-responsive cis-acting elements with the consensus sequence TGACGTGG/A (SEQ ID NO:XXX) (Wang, Y., et al. 2000. J. Biol. Chem. 275:27013-27020; Yoshida, H., et al. 2001. Cell 107:881-891). Such elements are suitable for use in the screening assays of the invention. As used herein, the term “ER stress” includes conditions such as the presence of reducing agents, depletion of ER lumenal Ca2+, inhibition of glycosylation or interference with the secretory pathway (by preventing transfer to the Golgi system), which lead to an accumulation of misfolded protein intermediates and increase the demand on the chaperoning capacity, and induce ER-specific stress response pathways. ER stress pathways involved with protein processing include the Unfolded Protein Response (UPR) and the Endoplasmic Reticulum Overload Response (EOR) which is triggered by certain of the same conditions known to activate UPR (e.g. glucose deprivation, glycosylation inhibition), as well as by heavy overexpression of proteins within the ER. The distinguishing feature of EOR is its association with the activation of the transcription factor NF-κB. Modulation of both the UPR and the EOR can be accomplished using the methods and compositions of the invention, ER stress can be induced, for example, by inhibiting the ER Ca2+ ATPase, e.g., with thapsigargin, As used herein, the term “protein folding or transport” encompasses posttranslational processes including folding, glycosylation, subunit assembly and transfer to the Golgi compartment of nascent polypeptide chains entering the secretory pathway, as well as extracytosolic portions of proteins destined for the external or internal cell membranes, that take place in the ER lumen. Proteins in the ER are destined to be secreted or expressed on the surface of a cell. Accordingly, expression of a protein on the cell surface or secretion of a protein can be used as indicators of protein folding or transport.

As used herein, the term “IRE-1” refers to an ER transmembrane endoribonuclease and kinase called inositol requiring enzyme, oligomerizes and is activated by autophosphorylation upon sensing the presence of unfolded proteins, see, e.g., Shamu et al., (1996) EMBO J. 15: 3028-3039. In Saccharomyces cerevisiae, the UPR is controlled by IREp. h1 the mammalian genome, there are two homologs of IRE-1, IRE1α and IRE1β. IRE1α is expressed in all cells and tissue whereas IRE1β is primarily expressed in intestinal tissue. The endoribonucleases of either IRE1α and IRE1β are sufficient to activate the UPR. Accordingly, as used herein, the term “IRE-1” includes, e.g., IRE1α, IRE1β and IREp. In a preferred embodiment, IRE-1 refers to IRE1α.

IRE-1 is a large protein having a transmembrane segment anchoring the protein to the ER membrane. A segment of the IRE-1 protein has homology to protein kinases and the C-terminal has some homology to RNAses. Over-expression of the IRE-1 gene leads to constitutive activation of the UPR. Phosphorylation of the IRE-1 protein occurs at specific serine or threonine residues in the protein.

IRE-1 senses the overabundance of unfolded proteins in the lumen of the ER. The oligomerization of this kinase leads to the activation of a C-terminal endoribonuclease by trans-autophosphorylation of its cytoplasmic domains. IRE-1 uses its endoribonuclease activity to excise an intron from XBP-1 mRNA. Cleavage and removal of a small intron is followed by re-ligation of the 5′ and 3′ fragments to produce a processed mRNA that is translated more efficiently and encodes a more stable protein (Calfon et al. (2002) Nature 415(3): 92-95). The nucleotide specificity of the cleavage reaction for splicing XBP-1 is well documented and closely resembles that for IRE-p mediated cleavage of HAC1 mRNA (Yoshida et al. (2001) Cell 107:881-891). In particular, IRE-1 mediated cleavage of murine XBP-1 cDNA occurs at nucleotides 506 and 532 and results in the excision of a 26 base pair fragment (e.g., CAGCACTCAGACTACGTGCACCTCTG (SEQ ID NO:1) for mouse XBP-1). IRE-1 mediated cleavage of XBP-1 derived from other species, including humans, occurs at nucleotides corresponding to nucleotides 506 and 532 of murine XBP-1 cDNA, for example, between nucleotides 502 and 503 and 528 and 529 of human XBP-1.

There are two transcript variants of human IRE-1, the sequence of which are known in the art and can be found at, e.g., at GenBank accession numbers: gi:50345998 and gi:153946420. The nucleotide and amino acid sequences of mouse and rat IRE-1 nay be found at, e.g., at GenBank accession numbers: gi:15284149 and gi:109489193, respectively.

XBP-1 controls expression of several other genes, for example, ERdj4, p58ipk, EDEM, PDI-P5, RAMP4, HEM BiP, ATF6α, XBP-1, Armet and DNAJB9, which encodes the 222 amino acid protein, mDj7 (GenBank Accession Number NM-013760 [gi:31560494]). These genes are important in a variety of cellular functions. For example, Hsp70 family proteins including BiP/Grp78 which is a representative ER localizing HSP70 member, function in protein folding in mammalian cells. A family of mammalian DnaJ/Hsp40-like proteins has recently been identified that are presumed to carry out the accessory folding functions. Two of them, Erdj4 and p58ipk, were shown to be induced by ER stress, localize to the ER, and modulate HSP70 activity (Chevalier et al. 2000 J Biol Chem 275: 19620-19627; Ohtsuka and Hata 2000 Cell Stress Chaperones 5: 98-112; Yan et al. 2002 Proc Natl Acad Sci USA 99: 15920-15925). ERdj4 has recently been shown to stimulate the ATPase activity of BiP, and to suppress ER stress-induced cell death (Kurisu et al. 2003 Genes Cells 8: 189-202; Shen et al. 2003 J Biol Chem 277: 15947-15956). ERdj4, p58IPK, EDEM, RAMP-4, PDI-P5 and HEDJ, all appear to act in the ER. ERdj4 (Shen et al, 2003), p58IPK (Melville et al. 1999 J Biol Chem 274: 3797-3803) and HEDJ (Yu et al. 2000 Mol Cell 6: 1355-1364) are localized to the ER and display Hsp40-like ATPase augmenting activity for the HspTO family chaperone proteins. EDEM was shown to be critically involved in the ERAD pathway by facilitating the degradation of ERAD substrates (Hosokawa et al. 2001 EMBO Rep 2:415-422; Molinari et al. 2003 Science 299 1397-1400; Oda et al. 2003 Science 299:1394-1397; Yoshida et al. 2003 Dev. Cell. 4:265-271), RAMP4 is a recently identified protein implicated in glycosylation and stabilization of membrane proteins in response to stress (Schroder et al. 1999 EMBO J 18:4804-4815; Wang and Dobberstein 1999 Febs Lett 457:316-322; Yamaguchi et al. 1999 J. Cell Biol 147:1195-1204). PDI-P5 has homology to protein disulfide isomerase, which is thought to be involved in disulfide bond formation (Kikuchi et al. 2002 J. Biochem (Tokyo) 132:451-455). Collectively, these results show that the IRE1/XBP-1 pathway is required for efficient protein folding, maturation and degradation in the ER.

Another UPR signaling pathway is activated by the PERK protein kinase, PERK phosphorylates eIF2α, which induces a transient suppression of protein translation accompanied by induction of transcription factor(s) such as ATF4 (Harding et al. 2000 Mol Cell 6: 1099-1108). eIF2α is also phosphorylated under various cellular stress conditions by specific kinases, double strand RNA activated protein kinase PKR, the amino acid control kinase GCN2 and the heme regulated inhibitor HR1 (Samuel 1993 J. Biol. Chem 268:7603-76-6; Kaufman 1999 Genes Dev. 13: 1211-1233). Since genes that are induced by the PERK pathway are also induced by other stress signals, such as amino acid deprivation, it is likely that PERK dependent UPR target genes carry out common cellular defense mechanisms, such as cellular homeostasis, apoptosis and cell cycle (Harding et al, 2003 Mol. Cell 11619-633). Collectively, ER stress activates IRE/XBP-1 and PERK/eIF2α pathways to ensure proper maturation and degradation of secretory proteins and to effect common cellular defense mechanisms, respectively.

The reliance of p58IPK gene expression on XBP-1 connects two of the UPR signaling pathways, IRE1/XBP-1 and PERK. P58IPK was originally identified as a 58 kD inhibitor of PKR in influenza virus-infected kidney cells (Lee et al. 1990 Proc Natl Acad Sci USA 87: 6208-6212) and described to downregulate the activity of PKR by binding to its kinase domain (Katze 1995 Trends Microbiol 3: 75-78). It also has a J domain in the C-terminus which has been shown to participate in interactions with Hsp70 family proteins Melville et al. 1999 J Biol Chem 274: 3797-380). Recently Katze and colleagues have demonstrated that p58IPK interacts with ERK which is structurally similar to PKR, inhibits its eIF2α kinase activity and that it is induced during the UPR by virtue of an ER stress-response element in its promoter region (Yan et al. 2002 Proc Natl Acad Sci USA 99: 15920-15925).

As used herein the term “activating transcription factors 6” include ATF6α and ATF6β. ATF6 is a member of the basic-leucine zipper family of transcription factors. It contains a transmembrane domain and is located in membranes of the endoplasmic reticulum. ATF6 is constitutively expressed in an inactive form in the membrane of the ER. Activation in response to ER stress results in proteolytic cleavage of its N-terminal cytoplasmic domain by the S2P serine protease to produce a potent transcriptional activator of chaperone genes (Yoshida et al. 1998 J. Biol. Chem. 273: 33741-33749; Li et al. 2000 Biochem J 350 Pt 1: 131-138; Ye et al. 2000 Mol Cell 6: 1355-1364; Yoshida et al. 2001 Cell 107: 881-891; Shen et al. 2002 Dev Cell 3: 99-111). The recently described ATF6β is closely related structurally to ATF6α and posited to be involved in the UPR (Haze et al. 2001 Biochem J 355: 19-28; Yoshida et al. 2001b Mol Cell Biol 21: 1239-1248). The third pathway acts at the level of posttranscriptional control of protein synthesis. An ER transmembrane component, PEK/PERK, related to PKR (interferon-induced double-stranded RNA-activated protein kinase) is a serine/threonine protein kinase that acts in the cytoplasm to phosphorylate eukaryotic initiation factor-2α (eIF2α). Phosphorylation of eIF2α results in translation attenuation in response to ER stress (Shi et al. 1998 Mol. Cell. Biol. 18: 7499-7509; Harding et al. 1999 Nature 397: 271-274).

The nucleotide and amino acid sequences of ATF6 are known in the art and can be found at, e.g., at GenBank accession number: gi:56786156, gi:124486810, gi:157821878 (human, mouse, rat, respectively).

As used herein, the various forms of the term “modulate” include stimulation (e.g., increasing or upregulating a particular response or activity) and inhibition (e.g., decreasing or downregulating a particular response or activity).

As used herein, the terms “a modulator of XBP-1” and “a modulator of IRE-1” include modulators of XBP-1 and/or IRE-1 expression, processing, post-translational modification, stability, and/or activity. The term includes agents, for example a compound or compounds which modulates transcription of, for example, an XBP-1 and/or IRE-1 gene, processing of an XBP-1 mRNA (e.g., splicing), translation of XBP-1 mRNA, post-translational modification of an XBP-1 and/or IRE-1 protein (e.g., glycosylation, ubiquitination), or activity of an XBP-1 and/or IRE-1 protein. In one embodiment, a modulator modulates one or more of the above. In preferred embodiments, the activity of XBP-1 and/or IRE-1 is modulated.

A “modulator of XBP-1 activity” and “a modulator of IRE-1 activity” include compounds that directly or indirectly modulate XBP-1 and/or IRE-1 activity. For example, an indirect modulator of XBP-1 activity can modulate a non-XBP-1 molecule which is in a signal transduction pathway that includes XBP-1. Examples of modulators that directly modulate XBP-1 expression, processing, post-translational modification, and/or activity include nucleic acid molecules encoding a biologically active portion of XBP-1, biologically active portions of XBP-1, antisense or siRNA nucleic acid molecules that bind to XBP-1 mRNA or genomic DNA, intracellular antibodies that bind to XBP-1 intracellularly and modulate (i.e., inhibit) XBP-1 activity, XBP-1 peptides that inhibit the interaction of XBP-1 with a target molecule (e.g., IRE-1) and expression vectors encoding XBP-1 that allow for increased expression of XBP-1 activity in a cell, dominant negative forms of XBP-1, as well as chemical compounds that act to specifically modulate the activity of XBP-1. Examples of modulators that directly modulate IRE-1 expression, processing, post-translational modification, and/or activity include nucleic acid molecules encoding a biologically active portion of IRE-1, biologically active portions of IRE-1, antisense or siRNA nucleic acid molecules that bind to IRE-1 mRNA or genomic DNA, IRE-1 peptides that inhibit the interaction of IRE-1 with a target molecule (e.g., XBP-1) and expression vectors encoding IRE-1 that allow for increased expression of IRE-1 activity in a cell, as well as chemical compounds that act to specifically modulate the activity of IRE-1.

As used interchangeably herein, the terms “XBP-1 activity,” “biological activity of XBP-1” or “functional activity XBP-1,” include activities exerted by XBP-1 protein on an XBP-1 responsive cell or tissue, e.g., a hepatocyte, a B cell, a macrophage, or on an XBP-1 nucleic acid molecule or protein target molecule, as determined in vivo, or in vitro, according to standard techniques. XBP-1 activity can be a direct activity, such as an association with an XBP-1-target molecule e.g., binding of spliced XBP-1 to a regulatory region of a gene responsive to XBP-1 (for example, a gene such as ERdj4, p58^{ipk}, EDEM, PDI-P5, RAMP4, HEDJ, BiP, ATF6α, XBP-1, Armet and/or DNAJB9), e.g., an unfolded protein response element (UPRE), or genes involved in de novo hepatic lipogenesis. Alternatively, an XBP-1 activity is an indirect activity, such as a downstream biological event mediated by interaction of the XBP-1 protein with an XBP-1 target molecule, e.g., EDEM. The biological activities of XBP-1 are described herein and include: e.g., modulation of TLR-mediated signaling, modulation of the UPR, modulation of IL-6 production, modulation of the proteasome pathway, modulation of protein folding and transport, modulation of apoptosis.

As used interchangeably herein, the terms “IRE-1 activity,” “biological activity of IRE-1” or “functional activity IRE-1,” include activities exerted by IRE-1 protein on an IRE-1 responsive cell or tissue, e.g., a hepatocyte, a B cell, or on an IRE-1 nucleic acid molecule or protein target molecule, as determined in vivo, or in vitro, according to standard techniques. IRE-1 activity can be a direct activity, such as an association with an IRE-1-target molecule e.g., XBP-1 phosphorylation of a substrate (e.g., autokinase activity) or endoribonuclease activity on a substrate e.g., XBP-1 mRNA. Alternatively, an IRE-1 activity is an indirect activity, such as a downstream biological event mediated by interaction of the IRE-1 protein with an IRE-1 target molecule, e.g., XBP-1. As IRE-1 is in a signal transduction pathway involving XBP-1, modulation of IRE-1 modulates a molecule in a signal transduction pathway involving XBP-1. Modulators which modulate an XBP-1 biological activity indirectly modulate expression and/or activity of a molecule in a signal transduction pathway involving XBP-1, e.g., IRE-1, PERK, eIF2α, or ATF6α. The biological activities of IRE-1 are described herein and include: e.g., modulation of TLR-mediated signaling, modulation of the UPR, modulation of IL-6 production, modulation of the proteasome pathway, modulation of protein folding and transport, modulation of apoptosis.

“Activity of unspliced XBP-1” includes the ability to modulate the activity of spliced XBP-1. In one embodiment, unspliced XBP-1 competes for binding to target DNA sequences with spliced XBP-1. In another embodiment, unspliced XBP-1 disrupts the formation of homodimers or heterodimers (e.g., with cfos or ATF6α) by XBP-1.

As used herein, a “substrate” or “target molecule” or “binding partner” is a molecule with which a protein binds or interacts in nature, such that that protein's function (e.g., modulation of TLR-mediated signaling, modulation of the UPR, and modulation of IL-6 production, modulation of the proteasome pathway, modulation of protein folding and transport, modulation of apoptosis) is achieved. For example, a target molecule can be a protein or a nucleic acid molecule. Exemplary target molecules of the invention include proteins in the same signaling pathway as the XBP-1 and/or IRE-1 protein, e.g., proteins which can function upstream (including both stimulators and inhibitors of activity) or downstream of the XBP-1 and/or IRE-1 protein in a pathway involving regulation of, for example, modulation of TLR-mediated signaling, modulation of ER stress, modulation of the UPR, modulation of IL-6 production, modulation of the proteasome pathway, modulation of protein folding and transport, modulation of apoptosis. Exemplary XBP-1 target molecules (based on their ability to interact with XBP-1) include IRE-1. XBP-1 itself (as the molecule forms homodimers), as well as the regulatory regions of genes regulated by XBP-1. Exemplary IRE-1 target molecules include XBP-1 and IRE-1 itself (as the molecule can form homodimers).

The subject methods can employ various target molecules. For example, in one embodiment, the subject methods employ XBP-1 or IRE-1. In another embodiment, the subject methods employ at least one other molecule, e.g., a molecule either upstream or downstream of XBP-1. For example, in one embodiment, the subject methods employ IRE-1. In another embodiment, the methods of the invention employ TRAF6.

“TRAF6” ialso referred to as “TNF receptor-associated factor 6” is a member of the TRAF family that is a signal transducer in the NF-kappa-B pathway that activates 1-kappa-B kinase in response to proinflammatory cytokines. There are two isoforms of human TRAF6, the nucleotide and amino acid sequences of which can be found at, for example, GenBank Accession No. gi:22027628 and gi:22027629. The nucleotide and amino acid sequences of mouse and rat TRAF6 can be found at, for example, GenBank Accession No. gi:38348245 and gi:197927342, respectively).

As used herein, the term “chaperone gene” is includes genes that are induced as a result of the activation of the UPR or the EOR. The chaperone genes include, for example, members of the family of Glucose Regulated Proteins (GRPs) such as GRP78 (BiP) and GRP94 (endoplasmin), as well as other chaperones such as calreticulin, protein disulfide isomerase, and ERp72. The upregulation of chaperone genes helps accommodate the increased demand for the folding capacity within the ER.

As used herein, the term “gene whose transcription is regulated by XBP-1”, includes genes having a regulatory region regulated by XBP-1. Such genes can be positively or negatively regulated by XBP-1. The term also includes genes which are indirectly regulated by XBP-1, e.g., are regulated by molecule in a signaling pathway in which XBP-1 is involved. Exemplary genes directly regulated by XBP-1 include, for example, lipogenic genes, e.g., fasn (gi:41872631, gi:93102409), proprotein convertase subtilisin/kexin type 9 (PCSK9) (gi:31317307, gi:23956352), stearyl coA desaturase (gi:53759151, gi:31543675), diacyl glycerol acetyltransferase 2 (gi:26024197, gi:16975490), acetyl coA carboxylase 2 (gi:134142062, gi:157042798), genes such as ERdj4 (e.g., NM-012328 [gi:9558754]), p58ipk (e.g., XM-209778 [gi:2749842] or NM-006260 [gi:24234721]), EDEM (e.g., NM-014674 [gi:7662001]), PDI-P5 (e.g., NC 003284 [gi:32566600]), RAMP4 (e.g., AF136975 [gi:12239332]), HEDJ (e.g., AF228505 [gi: 7385134]), BiP (e.g., X87949 [gi: 1143491]), ATF6α (e.g., NM-007348 [gi:6671584], XBP-1 (e.g., NM-005080 [gi:14110394]), Armet (e.g., NM-006010 [gi:51743920]) and/or DNAJB9 (which encodes mDj7) e.g., (NM-012328 [gi:9558754]), the MHC class II genes (various MHC class II gene sequences are known in the art) and the IL-6 gene (e.g., NM-000600 [gi 10834983], gi:13624310, gi:7549768 (human, mouse, rat, respectively)).

The term “interact” as used herein is meant to include detectable interactions between molecules, such as can be detected using, for example, a yeast two hybrid assay or coimmunoprecipitation. The term interact is also meant to include “binding” interactions between molecules. Interactions may be protein-protein or protein-nucleic acid in nature.

As used herein, the term “contacting” (i.e., contacting a cell e.g. a cell, with a compound) includes incubating the compound and the cell together in vitro (e.g., adding the compound to cells in culture) as well as administering the compound to a subject such that the compound and cells of the subject are contacted in vivo. The term “contacting” does not include exposure of cells to an XBP-1 and/or IRE-1 modulator that may occur naturally in a subject (i.e., exposure that may occur as a result of a natural physiological process).

As used herein, the term “test compound” refers to a compound that has not previously been identified as, or recognized to be, a modulator of the activity being tested. The term “library of test compounds” refers to a panel comprising a multiplicity of test compounds.

As used herein, the term “indicator composition” refers to a composition that includes a protein of interest (e.g., XBP-1), for example, a cell that naturally expresses the protein, a cell that has been engineered to express the protein by introducing an expression vector encoding the protein into the cell, or a cell free composition that contains the protein (e.g., purified naturally-occurring protein or recombinantly-engineered protein).

As used herein with respect to screening methods and methods of modulating innate immune responses, the term “cell” includes mammalian cells. In a preferred embodiment, a cell of the invention is a murine or human cell.

As used herein, the term “engineered” (as in an engineered cell) refers to a cell into which a nucleic acid molecule e.g., encoding an XBP-1 protein (e.g., a spliced and/or unspliced form of XBP-1) has been introduced.

As used herein, the term “cell free composition” refers to an isolated composition, which does not contain intact cells. Examples of cell free compositions include cell extracts and compositions containing isolated proteins.

As used herein, the term “reporter gene” refers to any gene that expresses a detectable gene product, e.g., RNA or protein. As used herein the term “reporter protein” refers to a protein encoded by a reporter gene. Preferred reporter genes are those that are readily detectable. The reporter gene can also be included in a construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall el al. (1983) J. Mol. Appl. Gen. 2: 101), human placental secreted alkaline phosphatase (Cullen and Malim (1992) Methods in Enzymol. 216:362-368) and green fluorescent protein (U.S. Pat. No. 5,491,084; WO 96/23898).

As used herein, the term “XBP-1-responsive element” refers to a DNA sequence that is directly or indirectly regulated by the activity of the XBP-1 (whereby activity of XBP-1 can be monitored, for example, via transcription of a reporter gene).

As used herein, the term “cells deficient in XBP-1” includes cells of a subject that are naturally deficient in XBP-1, as wells as cells of a non-human XBP-1 deficient animal, e.g., a mouse, that have been altered such that they are deficient in XBP-1. The term “cells deficient in XBP-1” is also intended to include cells isolated from a non-human XBP-1 deficient animal or a subject that are cultured in vitro.

As used herein, the term “non-human XBP-1 deficient animal” refers to a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal, such that the endogenous XBP-1 gene is altered, thereby leading to either no production of XBP-1 or production of a mutant form of XBP-1 having deficient XBP-1 activity. Preferably, the activity of XBP-1 is entirely blocked, although partial inhibition of XBP-1 activity in the animal is also encompassed. The term “non-human XBP-1 deficient animal” is also intended to encompass chimeric animals (e.g., mice) produced using a blastocyst complementation system, such as the RAG-2 blastocyst complementation system, in which a particular organ or organs (e.g., the lymphoid organs) arise from embryonic stem (ES) cells with homozygous mutations of the XBP-1 gene. The term “non-human XBP-1 deficient animal” is also intended to encompass animals (e.g., mice) that contain a conditional allele(s) of the XBP-1 gene, such as a cre-lox containing animal in which the XBP-1 gene is rendered non-functional following, e.g., mating of an animal containing a floxed allele with an animal containing a cre allele (cre recombinase, e.g., under the control of the Mx1 promoter), such as those described in, e.g., Lee, et al. (Science. 2008 Jun. 13; 320(5882):1492-6) or, Hetz, et al. (2008) Proc Natl Acad Sci, USA 105:757, the contents of each of which are incorporated herein by reference.

As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

In one embodiment, a nucleic acid molecule of the invention is an siRNA molecule. In one embodiment, a nucleic acid molecule of the invention mediates RNAi, RNA interference (RNAi) is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000), Tuschl, T. et al. Genes Dev. 13, 3191-3197 (1999); Cottrell T R, and Doering T L. 2003. Trends Microbiol. 11:37-43; Bushman F. 2003. Mol Therapy. 7:9-10; McManus M T and Sharp P A. 2002. Nat Rev Genet. 3:737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs or Ambion. In one embodiment one or more of the chemistries described herein or known in the art for use in antisense RNA can be employed in molecules that mediate RNAi.

An “isolated” nucleic acid molecule is one which is separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule (i.e., sequences located at the 5′ and 3′ ends of the nucleic acid molecule) in the genomic DNA of the organism from which the nucleic acid molecule is derived.

As used herein, an “isolated protein” or “isolated polypeptide” refers to a protein or polypeptide that is substantially free of other proteins, polypeptides, cellular material and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the XBP-1 protein is derived, or substantially free from chemical precursors or other chemicals when chemically synthesized. The language “substantially free of cellular material” includes preparations of XBP-1 protein in which the protein is separated from cellular components of the cells from which it is isolated or recombinantly produced.

The nucleic acids of the invention can be prepared, e.g., by standard recombinant DNA techniques. A nucleic acid of the invention can also be chemically synthesized using standard techniques, Various methods of chemically synthesizing polydeoxynucleotides are known, including solid-phase synthesis which has been automated in commercially available DNA synthesizers (See Itakura et at. U.S. Pat. No. 4,598,049; Caruthers et al. U.S. Pat. No. 4,458,066; and Itakura U.S. Pat. Nos. 4,401,796 and 4,373,071, incorporated by reference herein).

As used herein, the term “hybridizes under high stringency conditions” is intended to describe conditions for hybridization and washing under which nucleotide sequences having substantial homology (e.g., typically greater than 70% homology) to each other remain stably hybridized to each other. A preferred, non-limiting example of high stringency conditions are hybridization in a hybridization buffer that contains 6× sodium chloride/sodium citrate (SSC) at a temperature of about 45° C. for several hours to overnight, followed by one or more washes in a washing buffer containing 0.2×SSC, 0.1% SDS at a temperature of about 50-65° C.

The term “percent (%) identity” as used in the context of nucleotide and amino acid sequences (e.g., when one amino acid sequence is said to be X % identical to another amino acid sequence) refers to the percentage of identical residues shared between the two sequences, when optimally aligned. To determine the percent identity of two nucleotide or amino acid sequences, the sequences are aligned for optimal comparison purposes (e.g., gaps may be introduced in one sequence for optimal alignment with the other sequence). The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100).

Computer algorithms known in the art can be used to optimally align and compare two nucleotide or amino acid sequences to define the percent identity between the two sequences. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al. (1990) J. Mol. Biol. 215:403-10. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. For example, the nucleotide sequences of the invention were blasted using the default Blastn matrix 1-3 with gap penalties set at: existance 5 and extension 2. The amino acid sequences of the invention were blasted using the default settings: the Blosum62 matrix with gap penalties set at existance 11 and extension 1.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. If multiple programs are used to compare sequences, the program that provides optimal alignment (i.e., the highest percent identity between the two sequences) is used for comparison purposes.

In one embodiment, an XBP-1 and/or IRE-1 nucleic acid molecule has at least 50%, 55%, 60%, 65%, 70%, 75%, 80%, 85%, 90%, 95% nucleotide identity over the full length of the nucleotide sequences of human, mouse, or rat XBP-1 disclosed herein and/or known in the art.

In one embodiment, an XBP-1 and/or IRE-1 nucleic acid molecule has at least 50%, 55%, 60%, 65%, 70%, 75%, 80%, 85%, 90%, 95% nucleotide identity over the full length of the nucleotide sequences of human, mouse, or rat XBP-1 disclosed herein and/or known in the art and encodes a polypeptide with an XBP-1 biological activity as described herein.

In one embodiment, an XBP-1 and/or IRE-1 polypeptide has at least 50%, 55%, 60%, 65%, 70%, 75%, 80%, 85%, 90%, 95% amino acid identity over the full length of the amino acid sequence of human, mouse, or rat XBP-1 disclosed herein and/or known in the art.

In one embodiment, an XBP-1 and/or IRE-1 polypeptide has at least 50%, 55%, 60%, 65%, 70%, 75%, 80%, 85%, 90%, 95% amino acid identity over the full length of the amino acid sequences of human, mouse, or rat XBP-1 disclosed herein and/or known in the art and has an XBP-1 biological activity as described herein.

As used herein, the term “dominant negative” includes molecules, such as XBP-1 molecules (e.g., portions or variants thereof) that compete with native (i.e., wild-type) XBP-1 molecules, but which do not have XBP-1 activity. Such molecules effectively decrease XBP-1 activity in a cell.

As used herein, the term “antibody” is intended to include immunoglobulin molecules and immunologically active portions of immunoglobulin molecules, i.e., molecules that contain an antigen binding site which binds (immunoreacts with) an antigen, such as Fab and F(ab′)₂ fragments, single chain antibodies, intracellular antibodies, scFv, Fd, or other fragments, as well as intracellular antibodies. Preferably, antibodies of the invention bind specifically or substantially specifically to XBP-1 and/or IRE-1, molecules (i.e., have little to no cross reactivity with non-XBP-1 molecules). The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody compositions thus typically display a single binding affinity for a particular antigen with which it immunoreacts.

In one embodiment, small molecules can be used as test compounds. The term “small molecule” is a term of the art and includes molecules that are less than about 7500, less than about 5000, less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., Cane et al. 1998. Science 282:63), and natural product extract libraries. In another embodiment, the compounds are small, organic non-peptidic compounds. In a further embodiment, a small molecule is not biosynthetic. For example, a small molecule is preferably not itself the product of transcription or translation. In one embodiment, small molecule compounds are present on a microarray, see, e.g., Bradner J E, et al. 2006. Chem Biol. 13(5):493-504.

Various aspects of the present invention are described in further detail in the following subsections.

II. Methods of Treatment and/or Prevention

XBP-1 plays a key role in TLR-mediated signaling in cells of the innate immune system. Accordingly, the invention features methods for enhancing the TLR-mediated activation of a cell of the innate immune system or of enhancing an innate immune response in a subject. The claimed methods are not meant to include naturally occurring events. For example, the step of contacting includes administering the modulator in a treatment protocol and, in one embodiment the term “agent” or “modulator” is not meant to embrace endogenous mediators produced by the cells of a subject.

The subject methods employ agents that modulate XBP-1 expression, processing, post-translational modification, or activity (or the expression, processing, post-translational modification, or activity of another molecule in an XBP-1 signaling pathway (e.g., IRE-1) such that an XBP-1 and/or IRE-1 biological activity, e.g., TLR-mediated signaling is modulated.

In one embodiment, the methods and compositions of the invention can be used to modulate XBP-1 expression, processing, post-translational modification, and/or activity in a cell. In one embodiment, the cell is a mammalian cell. In another embodiment, the cell is a human cell. In one embodiment, the cell is a cell of the innate immune system. In one embodiment, the cell is a macrophage or a dendritic cell. Such modulation can occur in vitro or in vivo,

In one embodiment, cells in which, e.g., XBP-1, is modulated in vitro can be introduced, e.g., into an allogeneic subject, or e.g., reintroduced into a subject. In one embodiment, the invention also allows for modulation of XBP-1 in vivo, by administering to the subject an amount of a modulator of XBP-1 such that TLR-mediated signaling is modulated.

In one embodiment, a modulatory agent of the invention directly affects the expression, post-translational modification, and/or activity of XBP-1. In another embodiment, the expression of XBP-1 is modulated. In another embodiment, the post-translational modification of XBP-1 is modulated. In another embodiment, the activity of XBP-1 is modulated, e.g., TLR-mediated signaling. In one embodiment, the agent modulates the interaction of XBP-1 with a DNA molecule to which XBP-1 binds. In another embodiment, a modulatory agent of the invention indirectly affects the expression, post-translational modification, and/or activity of XBP-1.

The term “subject” is intended to include living organisms but preferred subjects are mammals. Examples of subjects include mammals such as, e.g., humans, monkeys, dogs, cats, mice, rats cows, horses, goats, and sheep.

Identification of compounds that modulate the biological effects of XBP-1 by directly or indirectly modulating XBP-1 activity allows for selective manipulation of these biological effects in a variety of clinical situations using the modulatory methods of the invention. For example, the stimulatory methods of the invention (i.e., methods that use a stimulatory agent) can result in increased expression, processing, post-translational modification, and/or activity of spliced XBP-1, which stimulates, e.g., TLR-mediated signaling. In another embodiment, the stimulatory methods of the invention can be used to increase the expression, processing, post-translational modification, and/or activity of a negative regulator of XBP-1 (e.g., unspliced XBP-1 or a dominant negative form of XBP-1) to inhibit e.g., TLR-mediated signaling.

In contrast, the inhibitory methods of the invention (i.e., methods that use an inhibitory agent) can inhibit the activity of spliced XBP-1 and inhibit, e.g., TLR mediated signaling.

In another embodiment, the inhibitory methods of the invention inhibit the activity of a negative regulator of XBP-1, e.g., unspliced XBP-1 or a dominant negative form of XBP-1. The XBP-1 unspliced protein is an example of an ubiquitinated and hence extremely unstable protein. XBP-1 spliced protein is not ubiquitinated, and has a much longer half life than unspliced XBP-1 protein. Proteasome inhibitors, for example, block ubiquitination, and hence stabilize XBP-1 unspliced but not spliced protein. Thus, the ratio of unspliced to spliced XBP-1 protein increases upon treatment with proteasome inhibitors. Since unspliced XBP-1 protein actually inhibits the function of the spliced protein, treatment with proteasome inhibitors blocks the activity of spliced XBP-1.

Thus, to treat and/or prevent a disorder wherein inhibition of a biological effect of spliced XBP-1 is desirable, such as a disorder that would benefit from reduced TLR-receptor-mediated signaling, an inhibitory method of the invention is selected such that spliced XBP-1 activity and/or expression is inhibited or a stimulatory method is selected which selectively stimulates the expression and/or activity of a negative regulator of XBP-1. Examples of disorders in which such inhibitory methods can be useful include unwanted inflammation.

Alternatively, to treat and/or prevent a disorder wherein stimulation of a biological effect of spliced XBP-1 is desirable, such as a disorder that would benefit from increased TLR-mediated signaling, a stimulatory method of the invention is selected such that spliced XBP-1 activity and/or expression is upregulated or an inhibitory method is selected such that the expression and/or activity of a negative regulator of XBP-1 is inhibited. Examples of disorders in which such stimulatory methods can be useful include situations in which an enhanced activation of the innate immune system is desired, e.g., in the case of natural infection or in the case of vaccination with an antigen or a nucleic acid molecule encoding an antigen.

Application of the modulatory methods of the invention for the prevention, treatment, and/or amelioration of at least one symptom, or normalization of at least one indicator of a disorder can result in curing the disorder, a decrease in at least one symptom associated with the disorder, either in the long term or short term (i.e., amelioration of the condition) or simply a transient beneficial effect to the subject.

The methods of modulating XBP-1 can be practiced either in vitro or in vivo. For practicing the method in vitro, cells can be obtained from a subject by standard methods and incubated (i.e., cultured) in vitro with a stimulatory or inhibitory compound of the invention to stimulate or inhibit, respectively, the activity of XBP-1, Methods for isolating cells are known in the art.

Cells treated in vitro with either a stimulatory or inhibitory compound can be administered to a subject to influence the biological effects of XBP-1. For example, cells can be isolated from a subject, expanded in number in vitro and the activity of, e.g., spliced XBP-1, activity in the cells using a stimulatory agent, and then the cells can be readministered to the same subject, or another subject tissue compatible with the donor of the cells. Accordingly, in another embodiment, the modulatory method of the invention comprises culturing cells in vitro with e.g., an XBP-1 modulator and further comprises administering the cells to a subject. For administration of cells to a subject, it may be preferable to first remove residual compounds in the culture from the cells before administering them to the subject. This can be done for example by gradient centrifugation of the cells or by washing of the tissue. For further discussion of ex vivo genetic modification of cells followed by readministration to a subject, see also U.S. Pat. No. 5,399,346 by W. F. Anderson et al.

In other embodiments, a stimulatory or inhibitory compound is administered to a subject in vivo. Such methods can be used to treat disorders, e.g., as detailed above. In one embodiment, a stimulatory or inhibitory compound is delivered directly to e.g. a cell of the innate immune system, e.g., a macrophage, using methods known in the art.

For stimulatory or inhibitory agents that comprise nucleic acids (e.g., recombinant expression vectors encoding, e.g., XBP-1; antisense RNA; or e.g., XBP-1 derived peptides), the compounds can be introduced into cells of a subject using methods known in the art for introducing nucleic acid (e.g., DNA) into cells. Examples of such methods include:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson el al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci, USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry el al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

In one embodiment, if the stimulatory or inhibitory compounds can be administered to a subject as a pharmaceutical composition. In one embodiment, the invention is directed to an active compound (e.g., a modulator of XBP-1) and a carrier. Such compositions typically comprise the stimulatory or inhibitory compounds, e.g., as described herein or as identified in a screening assay, e.g., as described herein, and a pharmaceutically acceptable carrier. Pharmaceutically acceptable carriers and methods of administration to a subject are described herein.

In one embodiment, the active compounds of the invention are administered in combination with other agents. For example, in one embodiment, an active compound of the invention, e.g., a compound that modulates an XBP-1 signal transduction pathway (e.g., by directly modulating XBP-1 activity) is administered with another compound known in the art to be useful in treatment of a particular condition or disease. For example, in one embodiment, an active compound of the invention (e.g., a compound that directly modulates XBP-1 activity) can be administered or in combination with an agent that induces ER stress in cells (e.g., an agent such as tunicamycin). In another embodiment, an active compound of the invention may be coadministered with an immunosuppressant (e.g., when decreased activation of the immune system is desired) or an adjuvant (e.g., when increased activation of the immune system is desired).

Compounds that can be used in the methods of the invention is described in further detail below.

A. Stimulatory Compounds

The methods of the invention using spliced XBP-1 stimulatory compounds can be used in the prevention and/or treatment of disorders in which spliced XBP activity and/or expression is undesirably reduced, inhibited, downregulated, or the like. Accordingly, preferred disorders for treatment using a stimulatory compound of the invention include, e.g., situations in which enhanced activation of cells of the innate immune system are desired or situations in which enhanced activation of the innate immune system are desired. In one embodiment, the stimulatory methods of the invention, a subject is treated with a stimulatory compound that stimulates expression and/or activity of spliced XBP-1. In another embodiment, a stimulatory method of the invention can be used to stimulate the expression and/or activity of a negative regulator of spliced XBP-1 activity.

Examples of stimulatory compounds include XBP-1 polypeptides, proteins, or biologically active fragments thereof, nucleic acid molecules encoding XBP-1 proteins or biologically active fragments thereof, and chemical agents that stimulate expression and/or activity of the protein of interest.

In one embodiment, stimulatory compound is a nucleic acid molecule encoding unspliced XBP-1 that is capable of being spliced or spliced XBP wherein the nucleic acid molecule is introduced into the subject in a form suitable for expression of the protein in the cells of the subject. For example, an XBP-1 cDNA (full length or partial cDNA sequence) is cloned into a recombinant expression vector and the vector is transfected into cells using standard molecular biology techniques. The XBP-1 cDNA can be obtained, for example, by amplification using the polymerase chain reaction (PCR) or by screening an appropriate cDNA library. The nucleotide sequences of XBP-1 cDNA are known in the art and can be used for the design of PCR primers that allow for amplification of a cDNA by standard PCR methods or for the design of a hybridization probe that can be used to screen a cDNA library using standard hybridization methods. Another preferred stimulatory compound is a nucleic acid molecule encoding the spliced form of XBP-1.

Following isolation or amplification of XBP-1 cDNA or cDNA encoding a molecule in a signal transduction pathway involving XBP-1, the DNA fragment is introduced into a suitable expression vector, as described above. For example, nucleic acid molecules encoding XBP-1 in the form suitable for expression of the XBP-1 in a host cell, can be prepared as described above using nucleotide sequences known in the art. The nucleotide sequences can be used for the design of PCR primers that allow for amplification of a cDNA by standard PCR methods or for the design of a hybridization probe that can be used to screen a cDNA library using standard hybridization methods.

In one embodiment, a stimulatory agent can be present in an inducible construct. In another embodiment, a stimulatory agent can be present in a construct which leads to constitutive expression.

Another form of a stimulatory compound for stimulating expression of XBP-1 or a molecule in a signal transduction pathway involving XBP-1 in a cell is a chemical compound that specifically stimulates the expression, processing, post-translational modification, or activity of endogenous spliced XBP-1 Such compounds can be identified using screening assays that select for compounds that stimulate the expression of XBP-1 that can be spliced or activity of spliced XBP-1 as described herein.

B. Inhibitory Compounds

The methods of the invention using inhibitory compounds which inhibit the expression, processing, post-translational modification, or activity of spliced XBP-1 can be used in the prevention and/or treatment of disorders in which spliced XBP-1 activity is undesirably enhanced, stimulated, upregulated or the like. For example, in situations where activation of the innate immune system is not desired, e.g., in cases of chronic inflammation or acute inflammation.

In a preferred embodiment, inhibitory compounds can be used to inhibit the expression, processing, post-translational modification, or activity of a negative regulator of XBP-1, e.g., unspliced XBP-1. Such compounds can be used in the treatment of disorders in which unspliced XBP-1 is undesirably elevated or when spliced XBP-1 expression and/or activity is undesirably reduced.

In one embodiment of the invention, an inhibitory compound can be used to inhibit (e.g., specifically inhibit) the expression, processing, post-translational modification, or activity of spliced XBP-1. Preferably, an inhibitory compound can be used to inhibit (e.g., specifically inhibit) the expression, processing, post-translational modification, or activity of unspliced XBP-1.

Inhibitory compounds of the invention can be, for example, intracellular binding molecules that act to specifically inhibit the expression, processing, post-translational modification, or activity e.g., of XBP-1 or a molecule in a signal transduction pathway involving XBP-1 (e.g., IRE-1). As used herein, the term “intracellular binding molecule” is intended to include molecules that act intracellularly to inhibit the processing expression or activity of a protein by binding to the protein or to a nucleic acid (e.g., an mRNA molecule) that encodes the protein. Examples of intracellular binding molecules, described in further detail below, include antisense nucleic acids, peptidic compounds that inhibit the interaction of XBP-1 with a target molecule and chemical agents that specifically inhibit XBP-1 activity.

i. Antisense or siRNA Nucleic Acid Molecules

In one embodiment, an inhibitory compound of the invention is an antisense nucleic acid molecule that is complementary to a gene encoding XBP-1, or to a portion of said gene, or a recombinant expression vector encoding said anti sense nucleic acid molecule. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:198-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the known nucleotide sequence for the coding strand of the XBP-1 gene and thus the known sequence of the XBP-1 mRNA, antisense nucleic acids of the invention can be designed according to the rules of Watson and Crick base pairing. The antisense nucleic acid molecule can be complementary to the entire coding region of an mRNA, but more preferably is antisense to only a portion of the coding or noncoding region of an mRNA. For example, the antisense oligonucleotide can be complementary to the region surrounding the translation start site of an XBP-1 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. To inhibit expression in cells, one or more antisense oligonucleotides can be used.

Alternatively, an antisense nucleic acid can be produced biologically using an expression vector into which all or a portion of a cDNA has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared according to standard recombinant DNA methods for constructing recombinant expression vectors, except that the cDNA (or portion thereof) is cloned into the vector in the antisense orientation. The antisense expression vector can be in the form of, for example, a recombinant plasmid, phagemid or attenuated virus. The antisense expression vector can be introduced into cells using a standard transfection technique.

The antisense nucleic acid molecules of the invention are typically administered to a subject or generated in situ such that they hybridize with or bind to cellular mRNA and/or genomic DNA encoding a protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, an antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid molecule of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation mRNAs. A ribozyme having specificity e.g., for an XBP-1, IRE-1, or ATF6α-encoding nucleic acid can be designed based upon the nucleotide sequence of the cDNA. For example, a derivative of a Tetrahyinena L-1 9 IVS RNA can be constructed in which the nucleotide sequence of the active site is complementary to the nucleotide sequence to be cleaved in, e.g., an XBP-1-encoding mRNA. See, e.g., Cech et al. U.S. Pat. No. 4,987,071 and Cech et al. U.S. Pat. No. 5,116,742. Alternatively, XBP-1 mRNA can be used to select a catalytic RNA having a specific ribonuclease activity from a pool of RNA molecules. See, e.g., Bartel, D. and Szostak, J. W. (1993) Science 261:1411-1418.

Alternatively, gene expression can be inhibited by targeting nucleotide sequences complementary to the regulatory region of a gene (e.g., an XBP-1 promoter and/or enhancer) to form triple helical structures that prevent transcription of a gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In another embodiment, a compound that promotes RNAi can be used to inhibit expression of XBP-1. RNA interference (RNAi is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999); Cottrell T R, and Doering T L. 2003. Trends Microbiol. 11:37-43; Bushman F. 2003. Mol Therapy. 7:9-10; McManus M T and Sharp P A. 2002. Nat Rev Genet. 3:737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabsor Ambion. In one embodiment one or more of the chemistries described above or known in the art for use in antisense RNA can be employed in molecules that mediate RNAi. A working example of XBP-1 specific RNAi in which an XBP-1-specific RNAi vector was constructed by inserting two complementary oligonucleotides for 5′-GGGATTCATGAATGGCCCTTA-3′ (SEQ ID NO:2) into the pBS/U6 vector.

Exemplary siRNA molecules specific for the unspliced form of murine XBP-1 are shown below:

Beginning at position 711:
Sense strand siRNA:
(SEQ ID NO.: 3)
GUUGGACCCUGUCAUGUUUtt
Antisense strand siRNA:
(SEQ ID NO.: 4)
AAACAUGACAGGGUCCAACtt
Beginning at position 853:
Sense strand siRNA:
(SEQ ID NO.: 5)
GCCAUUAAUGAACUCAUUCtt
Antisense strand siRNA:
(SEQ ID NO.: 6)
GAAUGAGUUCAUUAAUGGCtt

Exemplary siRNA molecules specific for the spliced form of murine XBP-1 are shown below:

Beginning at position 746:
Sense strand siRNA:
(SEQ ID NO.: 7)
GAAGAGAACCACAAACUCCUU
Antisense strand siRNA:
(SEQ ID NO.: 8)
GGAGUUUGUGGUUCUCUUCUU
Beginning at position 1307:
Sense strand siRNA:
(SEQ ID NO.: 9)
GAGGAUCACCCUGAAUUCAUU
Antisense strand siRNA:
(SEQ ID NO.: 10)
UGAAUUCAGGGUGAUCCUCUU

Exemplary siRNA molecules specific for the unspliced form of human XBP-1 are shown below:

Beginning at position 729:
Sense strand siRNA:
(SEQ ID NO.: 11)
CUUGGACCCAGUCAUGUUCUU
Antisense strand siRNA:
(SEQ ID NO.: 12)
GAACAUGACUGGGUCCAAGUU
Beginning at position 1079:
Sense strand siRNA:
(SEQ ID NO.: 13)
AUCUGCUUUCAUCCAGCCAUU
Antisense strand siRNA:
(SEQ ID NO.: 14)
UGGCUGGAUGAAAGCAGAUUU

Exemplary siRNA molecules specific for the spliced form of human XBP-1 are shown below:

Beginning at position 884:
Sense strand siRNA:
(SEQ ID NO.: 15)
GCCCCUAGUCUUAGAGAUAUU
Antisense strand siRNA:
(SEQ ID NO.: 16)
UAUCUCUAAGACUAGGGGCUU
Beginning at position 1108:
Sense strand siRNA:
(SEQ ID NO.: 17)
GAACCUGUAGAAGAUGACCUU
Antisense strand siRNA:
(SEQ ID NO.: 18)
GGUCAUCUUCUACAGGUUCUU.

Exemplary siRNA molecules specific for IRE-1 are shown below:

Beginning at position 345:
Sense strand siRNA: Sense strand siRNA:
(SEQ ID NO: 24)
UGAUGGCAGCCUGUAUACGUU
Antisense strand siRNA:
(SEQ ID NO.: 19)
CGUAUACAGGCUGCCAUCAUU
Beginning at position 1161:
Sense strand siRNA:
(SEQ ID NO.: 20)
CAAGCUCAACUACUUGAGGUU
Antisense strand siRNA:
(SEQ ID NO.: 21)
CCUCAAGUAGUUGAGCUUGUU.

ii. Peptidic Compounds

In another embodiment, an inhibitory compound of the invention is a peptidic compound derived from the XBP-1 amino acid sequence. For example, in one embodiment, the inhibitory compound comprises a portion of, e.g., XBP-1 (or a mimetic thereof) that mediates interaction of XBP-1 with a target molecule such that contact of XBP-1 with this peptidic compound competitively inhibits the interaction of XBP-1 with the target molecule.

The peptidic compounds of the invention can be made intracellularly in cells by introducing into the cells an expression vector encoding the peptide. Such expression vectors can be made by standard techniques using oligonucleotides that encode the amino acid sequence of the peptidic compound. The peptide can be expressed in intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like).

In addition, dominant negative proteins (e.g., of XBP-1) can be made which include XBP-1 molecules (e.g., portions or variants thereof) that compete with native (i.e., wild-type) molecules, but which do not have the same biological activity. Such molecules effectively decrease, e.g., XBP-1 activity in a cell. For example, the peptide compound can be lacking part of an XBP-1 transcriptional activation domain, e.g., can consist of the portion of the N-terminal 136 or 188 amino acids of the spliced form of XBP-1.

iii. Other Agents that Act Upstream of XBP-1

In one embodiment, the expression of spliced XBP-1 can be inhibited using an agent that inhibits a signal that increases XBP-1 expression, processing, post-translational modification or activity in a cell. Both IL-4 and IL-6 have been shown to increase transcription of XBP-1 (Wen et al. 1999, Int. Journal of Oncology 15:173; Iwakoshi, et al. (2003) Nat. Immunol. 4 (4): 321-9). Accordingly, in one embodiment, an agent that inhibits a signal transduced by IL-4 or IL-6 can be used to downmodulate XBP-1 expression and, thereby, decrease the activity of spliced XBP-1 in a cell. For example, in one embodiment, an agent that inhibits a STAT-6 dependent signal can be used to decrease the expression of XBP-1 in a cell.

Other inhibitory agents that can be used to specifically inhibit the activity of an XBP-1 or a molecule in a signal transduction pathway involving XBP-1 are chemical compounds that directly inhibit expression, processing, post-translational modification, and/or activity of, e.g., an XBP-1 target protein activity or inhibit the interaction between, e.g., XBP-1 and target molecules. Such compounds can be identified using screening assays that select for such compounds, as described in detail above as well as using other art recognized techniques.

III. Pharmaceutical Compositions

A pharmaceutical composition comprising a compound of the invention, e.g., a stimulatory or inhibitory molecule of the invention or a compound identified in the subject screening assays, is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid; buffers such as acetates, citrates or phosphates and compounds for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition will preferably be sterile and should be fluid to the extent that easy syringability exists. It will preferably be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition an compound which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

In one embodiment, the test compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In one embodiment, a modulatory agent of the invention is administered in amount sufficient to modulate TLR-mediated signaling, e.g., such that the activation of a cell of the innate immune system is modulated. Such indicators may be measured using methods known in the art to measure the activation of macrophages or dendritic cells.

IV. Screening Assays

In one embodiment, the invention provides methods (also referred to herein as “screening assays”) for identifying agents for modulating TLR-mediated signaling by modulating XBP-1 activity. The subject assays generally involve testing the effect of a candidate agent on TLR-mediated signaling using methods known in the art or described herein. In one embodiment, the subject assays further comprise a step in which the effect of the agent on another activity of XBP-1 or on an activity or IRE-1 is measured, the ability to bind to XBP-1 is measured (e.g., in vitro or in silico), or an effect on the expression, processing (e.g., splicing), post-translational modification (e.g., glycosylation, ubiquitination, phosphorylation, or stability) of XBP-1 and/or IRE-1 alpha is measured.

In one embodiment, the ability of a compound to modulate TLR-mediated signaling is measured in a screening assay of the invention. In another embodiment, the ability of a compound to directly modulate the expression, processing (e.g., splicing), post-translational modification (e.g., glycosylation, ubiquitination, or phosphorylation), stability or activity of XBP-1 and/or IRE-1 alpha is measured in a screening assay of the invention.

The indicator composition can be a cell that expresses the XBP-1 and/or IRE-1 alpha protein, for example, a cell that naturally expresses or, more preferably, a cell that has been engineered to express the protein by introducing into the cell an expression vector encoding the protein. Preferably, the cell is a mammalian cell, e.g., a human cell. In another embodiment, the cell is a cell of the innate immune system, e.g., a hematopoietic cell. In one embodiment, the cell is a macrophage or a dendritic cell. Alternatively, the indicator composition can be a cell-free composition that includes the protein (e.g., a cell extract or a composition that includes e.g., either purified natural or recombinant protein). In one embodiment, the cell is under ER stress. In another embodiment, the cell is stimulated with a TLR agonist, e.g., lipopolysaccharide (LPS), lipoteichoic acid, PAM3CSK4, and FSL1.

Compounds identified as upmodulating the expression, activity, and/or stability of spliced XBP-1 and/or the expression and/or activity of IRE-1, and/or TLR-mediated signaling using the assays described herein are useful for modulating activation in cells of the innate immune system.

The subject screening assays can be performed in the presence or absence of other agents. In one embodiment, the subject assays are performed in the presence of an agent that affects the unfolded protein response, e.g., tunicamycin, which evokes the UPR by inhibiting N-glycosylation, or thapsigargin. In another embodiment, the subject assays are performed in the presence of an agent that inhibits degradation of proteins by the ubiquitin-proteasome pathway (e.g., peptide aldehydes, such as MG132). In another embodiment, the screening assays can be performed in the presence or absence of a molecule that enhances cell activation.

In another aspect, the invention pertains to a combination of two or more of the assays described herein. For example, an agent that modulates TLR-mediated signaling can be identified using a cell-based assay, and the ability of the agent to modulate the activity of XBP-1 or a molecule in a signal transduction pathway involving XBP-1 can be determined as described herein and in, for example, PCT/US2003/027404 and PCT/US2007/020658, the contents of which are expressely incorporated herein by reference.

Similarly, a modulating agent can be identified using a cell-based or a cell-free assay, and the ability of the agent to modulate the activity of XBP-1 or a molecule in a signal transduction pathway involving XBP-1 can be confirmed in vivo, e.g., in an animal model for immune cell activation or inflammation.

For example, in one embodiment, a modulating agent identified using the cell-based or cell-free assays described herein may be assayed in a non-human animal model of F. tularensis subsp. holarctica Live Vaccine Strain (LVS) infection. Such methods generally comprise administering the test compound to the non-human animal and determining the effect of the agent on, for example, cytokine production and/or survival.

Furthermore, a modulating agent may be identified in silico and the ability of the agent to modulate XBP-1 activity, IRE-1 activity, TLR-mediated signaling, and/or endoplasmic reticulum (ER) stress, may be further evaluated. For example, a program such as DOCK can be used to identify molecules which will bind to XBP-1 and/or IRE-1 and such molecules may be further evaluated as described herein.

In another embodiment, a modulating agent may be identified as modulating, e.g., decreasing the amount of XBP-1s protein and/or the ratio of spliced to upspliced XBP-1 mRNA and/or protein, and/or XBP-1 and/or IRE-1 activity and the ability of the agent to modulate TLR-mediated signaling, and/or endoplasmic reticulum (ER) stress, and/or the unfolded protein response, may be further evaluated.

In addition, a modulating agent can be identified using a cell-based or a cell-free assay, and the ability of the agent to modulate XBP-1 activity, IRE-1 activity, TLR-mediated signaling, and/or endoplasmic reticulum (ER) stress, and/or the uncoupled protein response, may be further evaluated. For example, in one embodiment, a modulating agent of interest may be further assayed for the ability to modulate the UPR using, for example, a UPR reporter assay. In another embodiment, a modulating agent of interest may be further assayed for the ability to modulate XBP-1 splicing (e.g., in HeLa cells or macrophages). An agent of interest may be assayed to determine whether the compound modulates XBP-1 protein synthesis. In addition, an agent of inerest may be assayed to determine whether the compound modulates IL-6 production using, e.g., RAW cells. In another embodiment, a compound of interest may be further tested to determine whether the compound also modulates immunoglobulin production, e.g., LPS-driven antibody production in primary cells, (e.g., isolated B cells), or cell lines, e.g., human SKW5.2 B lymphoma, mouse BCL1 B lymphoma.

Furthermore, a modulating agent may be identified in silico and the ability of the agent to modulate XBP-1 activity, IRE-1 activity, TLR-mediated signaling, and/or endoplasmic reticulum (ER) stress, and/or the uncoupled protein response, may be further evaluated. For example, a program such as DOCK can be used to identify molecules which will bind to XBP-1 and/or 1RE-1 and such molecules may be further evaluated as described herein. See DesJarlias et al. (1988) J. Med. Chem. 31:722; Meng et al. (1992) J. Computer Chem. 13:505; Meng et al. (1993) Proteins 17:266; Shoichet et al. (1993) Science 259:1445.

In another embodiment, a modulating agent may be identified as modulating, e.g., decreasing the amount of XBP-1s protein and/or the ratio of spliced to upspliced XBP-1 mRNA and/or protein, and/or XBP-1 and/or IRE-1 activity and the ability of the agent to modulate TLR-mediated signaling, and/or endoplasmic reticulum (ER) stress, and/or the uncoupled protein response, may be further evaluated.

Moreover, a modulator, identified as described herein (e.g., an enzyme, an antisense nucleic acid molecule, or a specific antibody, or a small molecule) can be used in an animal model to determine the efficacy, toxicity, or side effects of treatment with such a modulator. Alternatively, a modulator identified as described herein can be used in an animal model to determine the mechanism of action of such a modulator.

In another embodiment, it will be understood that similar screening assays can be used to identify compounds that indirectly modulate the activity and/or expression of XBP-1 and/or IRE-1 alpha, e.g., by performing screening assays such as those described above using molecules with which XBP-1 interacts, e.g., molecules that act either upstream or downstream of XBP-1 in a signal transduction pathway.

The cell based and cell free assays of the invention are described in more detail below.

A. Cell Based Assays

The indicator compositions of the invention can be a cell that expresses an XBP-1 and/or IRE-1 alpha protein, for example, a cell that naturally expresses endogenous XBP-1 or, more preferably, a cell that has been engineered to express an exogenous XBP-1 protein by introducing into the cell an expression vector encoding the protein. Alternatively, the indicator composition can be a cell-free composition that includes XBP-1 or a non-XBP-1 protein such as IRE-1, or a composition that includes purified XBP-1 or IRE-1.

Compounds that modulate expression and/or activity of XBP-1 and/or IRE-1 alpha and/or modulation of TLR-mediated signaling, modulation of ER stress, modulation of the UPR, modulation of IL-6 production, modulation of the proteasome pathway, modulation of protein folding and transport, modulation of apoptosis, can be identified using various “read-outs.” In one embodiment, during the detecting step, one or more components is transformed (e.g., by labeling).

For example, an indicator cell can be transfected with an XBP-1 expression vector, incubated in the presence and in the absence of a test compound, and the effect of the compound on the expression of the molecule or on a biological response regulated by XBP-1 and/or modulation of TLR-mediated signaling, modulation of ER stress, modulation of the UPR, modulation of IL-6 production, modulation of the proteasome pathway, modulation of protein folding and transport, modulation of apoptosis can be determined. In one embodiment, unspliced XBP-1 (e.g., capable of being spliced so that the cell will make both forms, or incapable of being spliced so the cell will make only the unspliced form) can be expressed in a cell. In another embodiment, spliced XBP-1 can be expressed in a cell. The biological activities of XBP-1 include activities determined in vivo, or in vitro, according to standard techniques. An XBP-1 activity can be a direct activity, such as an association with an XBP-1-target molecule. Alternatively, an XBP-1 activity is an indirect activity, such as a cellular signaling activity or alteration in gene expression occurring downstream of the interaction of the XBP-1 protein with an XBP-1 target molecule or a biological effect occurring as a result of the signaling cascade triggered by that interaction. For example, biological activities of XBP-1 described herein include: modulation of TLR-mediated signaling, modulation of ER stress, modulation of the UPR, modulation of proinflammatory cytokine production, e.g., modulation of IL-6 production, e.g., sustained production, modulation of IFNβ production, e.g., sustained production, modulation of ISG15 production, e.g., sustained production, modulation of the proteasome pathway, modulation of protein folding and transport, modulation of apoptosis.

The biological activities of IRE-1 alpha include activities determined in vivo, or in vitro, according to standard techniques. An IRE-1 alpha activity can be a direct activity, such as an association with an IRE-1 alpha-target molecule (e.g., a nucleic acid molecule to which IRE-1 alpha binds or a protein such as the XBP-1 protein. Alternatively, an IRE-1 alpha activity is an indirect activity, such as a cellular signaling activity or alteration in gene expression occurring downstream of the interaction of the IRE-1 alpha protein with an IRE-1 alpha target molecule or a biological effect occurring as a result of the signaling cascade triggered by that interaction. For example, biological activities of IRE-1 alpha described herein include: modulation of TLR-mediated signaling, modulation of ER stress, modulation of the UPR, modulation of proinflammatory cytokine production, e.g., modulation of IL-6 production, e.g., sustained production, modulation of IFNβ production, e.g., sustained production, modulation of ISG15 production, e.g., sustained production, modulation of the proteasome pathway, modulation of protein folding and transport, modulation of apoptosis.

In another embodiment, the invention provides methods for identifying compounds that modulate cellular responses in which XBP-1 and/or IRE-1 is involved. For example, in one embodiment, modulation of the UPR can be determined and used as an indicator of modulation of XBP-1 and/or IRE-1 activity.

For example, to determine whether a test compound modulates TLR-mediated signaling, the effect of a test compound on XBP-1 and/or IRE-1 alpha activity may be determined. The effect of a test compound on TLR-mediated signaling may also be determined by determining the effect on cytokine, e.g., proinflammatory cytokine production, activation of TLR adaptor molecules, e.g., MyD88, TRIF, the activation of TRAF6, NADPH oxidase. To determine whether a test compound modulates activation of TLR adaptor molecules, e.g., MyD88, TRIF, the activation of TRAF6, NADPH oxidase, the effect of a test compound in the presence and absence of a selective inhibitor, e.g., an shRNA molecule, or pharmacological inhibitors, may be determined.

To determine whether a test compound modulates XBP-1 and/or IRE-1 protein expression, in vitro transcriptional assays can be performed. In one example of such an assay, the full length XBP-1 gene or promoter and enhancer of XBP-1 operably linked to a reporter gene such as chloramphenicol acetyltransferase (CAT) or luciferase and introduced into host cells. The expression or activity of XBP-1 or the reporter gene can be measured using techniques known in the art. The ability of a test compound to regulate the expression or activity of a molecule in a signal transduction pathway involving XBP-1 can be similarly tested.

As used interchangeably herein, the terms “operably linked” and “operatively linked” are intended to mean that the nucleotide sequence is linked to a regulatory sequence in a manner which allows expression of the nucleotide sequence in a host cell (or by a cell extract).

In another embodiment, modulation of expression of a protein whose expression is regulated by XBP-1 is measured. Regulatory sequences are art-recognized and can be selected to direct expression of the desired protein in an appropriate host cell. The term regulatory sequence is intended to include promoters, enhancers, polyadenylation signals and other expression control elements. Such regulatory sequences are known to those skilled in the art and are described in Goeddel, Gene Expression Technology: Methods in Enzymology 185, Academic Press, San Diego, Calif. (1990). It should be understood that the design of the expression vector may depend on such factors as the choice of the host cell to be transfected and/or the type and/or amount of protein desired to be expressed.

Exemplary constructs can include, for example, an XBP-1 target sequence TGGATGACGTGTACA fused to the minimal promoter of the mouse RANTES gene (Clauss et al. Nucleic Acids Research 1996. 24:1855) or the ATF6/XBP-1 target TCGAGACAGGTGCTGACGTGGCGATTCC and comprising −53/+45 of the cfos promoter (J. Biol. Chem, 275:27013) fused to a reporter gene. In one embodiment, multiple copies of the XBP-1 target sequence can be included.

In another embodiment, to determine whether a test compound modulates modulation of TLR-mediated signaling, modulation of ER stress, modulation of the UPR, modulation of IL-6 production, modulation of the proteasome pathway, modulation of protein folding and transport, modulation of apoptosis and/or XBP-1 activity, a test compound may be assayed by determining the effect of the compound on the ability of XBP-1 to transactivate a reporter gene. For example, a recombinant expression vector comprising a DNA binding region of e.g., a GAL4 protein (e.g., amino acids 1-147), can be operably linked to XBP-1 or a fragment thereof, e.g., the transactivation domain, e.g., amino acid residues 159-371 of spliced human XBP-1 protein, and the effect of a test compound can be assayed by determining whether XBP-1 can transactivate a reporter construct comprising e.g., a regulatory element responsive to the DNA binding region, e.g., a promoter or consensus binding sites(s) of e.g., GAL4 operably linked to a reporter gene, e.g., luciferase.

A variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard methods for measuring the activity of these gene products are known in the art.

A variety of cell types are suitable for use as an indicator cell in the screening assay. Preferably a cell line is used which expresses low levels of endogenous XBP-1 and/or IRE-1, and is then engineered to express recombinant XBP-1 and/or IRE-1. Cells for use in the subject assays include both eukaryotic and prokaryotic cells. For example, in one embodiment, a cell is a bacterial cell. In another embodiment, a cell is a fungal cell, such as a yeast cell. In another embodiment, a cell is a vertebrate cell, e.g., an avian cell or a mammalian cell (e.g., a murine cell, or a human cell).

In one embodiment, the level of expression of the reporter gene in the indicator cell in the presence of the test compound is higher than the level of expression of the reporter gene in the indicator cell in the absence of the test compound and the test compound is identified as a compound that stimulates the expression of the molecule. In another embodiment, the level of expression of the reporter gene in the indicator cell in the presence of the test compound is lower than the level of expression of the reporter gene in the indicator cell in the absence of the test compound and the test compound is identified as a compound that inhibits the expression of the molecule.

In another embodiment, modulation of the UPR or ER stress can also be determined and used as an indicator of modulation of XBP-1 and/or IRE-1 activity. Transcription of genes encoding molecular chaperones and folding enzymes in the endoplasmic reticulum (ER) is induced by accumulation of unfolded proteins in the ER. This intracellular signaling, known as the unfolded protein response (UPR), is mediated by the cis-acting ER stress response element (ERSE) or unfolded protein response element (UPRE) in mammals.

The activation of the kinase PERK can also be measured to determine whether an agent moduleates ER stress by measuring the induction of CHOP. The processing of ATF6 alpha can also be measured to determine whether an agent modulates ER stress. The basic leucine zipper protein ATF6 alpha isolated as a CCACG-binding protein is synthesized as a transmembrane protein in the ER, and ER stress-induced proteolysis produces a soluble form of ATF6 alpha that translocates into the nucleus.

In one embodiment, compounds that modulate TLR-mediated signaling and do not activate ER stress and/or the UPR are identified. In another embodiment, compounds that modulate TLR-mediated signaling and do activate ER stress and/or the UPR are identified.

In one embodiment, modulation of XBP-1 activity can be measured by determining the phosphorylation status of IRE-1, PERK or eIF2α, e.g., using commercially available antibodies that specifically recognize phosphorylated forms of the proteins. Increased phosphorylation of these molecules is observed under conditions of ER stress and the UPR.

In one embodiment differentiation of cells can be used as an indicator of modulation of XBP-1 or a signal transduction pathway involving XBP-1. Cell differentiation can be monitored directly (e.g. by microscopic examination of the cells for monitoring cell differentiation), or indirectly, e.g., by monitoring one or more markers of cell differentiation (e.g., an increase in mRNA for a gene product associated with cell differentiation, or the secretion of a gene product associated with cell differentiation, such as the secretion of a protein (e.g., the secretion of immunoglobulin by differentiated plasma cells) or the expression of a cell surface marker (such as Syndecan expression by plasma cells) Reimold et al. 2001. Nature 412:300). Standard methods for detecting mRNA of interest, such as reverse transcription-polymerase chain reaction (RT-PCR) and Northern blotting, are known in the art. Standard methods for detecting protein secretion in culture supernatants, such as enzyme linked immunosorbent assays (ELISA), are also known in the art. Proteins can also be detected using antibodies, e.g., in an immunoprecipitation reaction or for staining and FACS analysis.

In one embodiment, the ability of a compound to induce terminal B cell differentiation can be determined. Terminal B cell differentiation can be measured in a variety of ways. Cells can be examined microscopically for the presence of the elaborate ER system characteristic of plasma cells. The secretion of immunoglobulin is a hallmark of plasma cell differentiation.

In one embodiment, the ability of a compound to modulate proinflammatory cytokine production, e.g., IL-6, IFNβ, ISG15, can be determined. Production of proinflammatory cytokine can be monitored, for example, using RT-PCR, Northern or

Western blotting. Proinflammatory cytokine can also be detected using an ELISA assay or in a bioassay, e.g., employing cells which are responsive to proinflammatory cytokine (e.g., cells which proliferate in response to the cytokine or which survive in the presence of the cytokine), such as plasma cells or multiple myeloma cells using standard techniques.

In one embodiment, the effect of a test compound on sustained production of a proinflammatory cytokine may be determined. For example, the production of a proinflammatory cytokine may be determined at multiple time points, e.g., a time course assay, e.g., at about 0, 0.5, 1. 1.5, 2, 2.5, 3, 3.5, 4, 4.5, 5. 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 24 hours, following exposure to a test compound.

In another embodiment, the ability of a compound to modulate the proteasome pathway of a cell can be determined using any of a number of art-recognized techniques. For example, in one embodiment, the half life of normally short-lived regulatory proteins (e.g., NF-κB, cyclins, oncogenic products or tumor suppressors) can be measured to measure the degradation capacity of the proteasome. In another embodiment, the presentation of antigen in the context of MHC molecules on the surface of cells can be measured (e.g., in an in vitro assay of T cell activation) as proteasome degradation of antigen is important in antigen processing and presentation. In another embodiment, threonine protease activity associated with the proteasome can be measured. Agents that modulate the proteasome pathway will affect the normal degradation of these proteins.

In another embodiment, the modulation of the proteasome pathway can be measured indirectly by measuring the ratio of spliced to unspliced XBP-1 or the ratio of unspliced to spliced XBP-1. Inhibition of the proteasome pathway, e.g., by the inhibitor MG-132, leads to an increase in the level of unspliced XBP-1 as compared to spliced XBP-1.

The techniques for assessing the ratios of unspliced to spliced XBP-1 and spliced to unspliced XBP-1 are routine in the art. For example, the two forms can be distinguished based on their size, e.g., using northern blots or western blots. Because the spliced form of XBP-1 comprises an exon not found in the unspliced form, in another embodiment, antibodies that specifically recognize the spliced or unspliced form of XBP-1 can be developed using techniques well known in the art (Yoshida et al. 2001. Cell. 107:881). In addition, PCR can be used to distinguish spliced from unspliced XBP-1. For example, as described herein, primer sets can be used to amplify XBP-1 where the primers are derived from positions 410 and 580 of murine XBP-1, or corresponding positions in related XBP-1 molecules, in order to amplify the region that encompasses the splice junction. A fragment of 171 base pairs corresponds to unspliced XBP-1 mRNA. An additional band of 145 bp corresponds to the spliced form of XBP-1. The ratio of the different forms of XBP-1 can be determined using these or other art recognized methods.

Compounds that alter the ratio of unspliced to spliced XBP-1 or spliced to unspliced XBP-1 can be useful to modulate TLR-mediated signaling and/or the activity of XBP-1, and the levels of these different forms of XBP-1 can be measured using various techniques described above, or known in the art, and a ratio determined.

In one embodiment, the ability of a compound to modulate protein folding or transport can be determined. The expression of a protein on the surface of a cell or the secretion of a secreted protein can be measured as indicators of protein folding and transport. Protein expression on a cell can be measured, e.g., using FACS analysis, surface iodination, immunoprecipitation from membrane preparations. Protein secretion can be measured, for example, by measuring the level of protein in a supernatant from cultured cells. The production of any secreted protein can be measured in this manner. The protein to be measured can be endogenous or exogenous to the cell. The production of proteins can be measured using standard techniques in the art.

In another embodiment, the ability of a compound to modulate apoptosis, e.g., modulate apoptosis by disrupting the UPR, can be determined. In one embodiment, the ability of a compound to modulate apoptosis in a secretory cell or a cell under ER stress is determined. In one embodiment, cytochrome C release from mitochondria during cell apoptosis can be detected, e.g., plasma cell apoptosis (as described in, for example, Bossy-Wetzel E. et al. (2000) Methods in Enzymol. 322:235-42). Other exemplary assays include: cytofluorometric quantitation of nuclear apoptosis induced in a cell-free system (as described in, for example, Lorenzo H. K. et al. (2000) Methods in Enzymol. 322:198-201); apoptotic nuclease assays (as described in, for example, Hughes F. M. (2000) Methods in Enzymol. 322:47-62); analysis of apoptotic cells, e.g., apoptotic plasma cells, by flow and laser scanning cytometry (as described in, for example, Darzynkiewicz Z. et al. (2000) Methods in Enzymol. 322:18-39); detection of apoptosis by annexin V labeling (as described in, for example, Bossy-Wetzel E. et al. (2000) Methods in Enzymol. 322:15-18); transient transfection assays for cell death genes (as described in, for example, Miura M. et al. (2000) Methods in Enzymol. 322:480-92); and assays that detect DNA cleavage in apoptotic cells, e.g., apoptotic plasma cells (as described in, for example, Kauffman S. H. et al. (2000) Methods in Enzymol. 322:3-15), Apoptosis can also be measured by propidium iodide staining or by TUNEL assay. In another embodiment, the transcription of genes associated with a cell signaling pathway involved in apoptosis (e.g., JNK, caspases) can be detected using standard methods.

In yet another embodiment, the ability of a compound to modulate translocation of spliced XBP-1 to the nucleus can be determined.

In another embodiment, the activation of NFκB can be determined by determining the ability of a compound to modulate translocation of spliced NFκB to the nucleus.

Translocation of spliced XBP-1 to the nucleus can be measured, e.g., by nuclear translocation assays in which the emission of two or more fluorescently-labeled species is detected simultaneously. For example, the cell nucleus can be labeled with a known fluorophore specific for DNA, such as Hoechst 33342.

The spliced XBP-1 protein can be labeled by a variety of methods, including expression as a fusion with GFP or contacting the sample with a fluorescently-labeled antibody specific for spliced XBP-1. The amount of spliced XBP-1 that translocates to the nucleus can be determined by determining the amount of a first fluorescently-labeled species, i.e., the nucleus, that is distributed in a correlated or anti-correlated manner with respect to a second fluorescently-labeled species, i.e., spliced XBP-1, as described in U.S. Pat. No. 6,400,487, the contents of which are hereby incorporated by reference.

In another embodiment, the ability of XBP-1 and/or IRE-1 to be acted on by an enzyme or to act on a substrate can be measured. For example, in one embodiment, the effect of a compound on the phosphorylation of IRE-1, the ability of IRE-1 to process XBP-1, the ability of PERK to phosphorylate a substrate can be measured using techniques that are known in the art.

The ability of the test compound to modulate XBP-1 and/or IRE-1 binding to a substrate or target molecule can also be determined. Determining the ability of the test compound to modulate XBP-1 (or IRE-1) binding to a target molecule (e.g., a binding partner such as a substrate) can be accomplished, for example, by coupling the target molecule with a radioisotope or enzymatic label such that binding of the target molecule to XBP-1 can be determined by detecting the labeled XBP-1 (or IRE-1) target molecule in a complex. Alternatively, XBP-1 could be coupled with a radioisotope or enzymatic label to monitor the ability of a test compound to modulate XBP-1 binding to a target molecule in a complex. Determining the ability of the test compound to bind to XBP-1 can be accomplished, for example, by coupling the compound with a radioisotope or enzymatic label such that binding of the compound to XBP-1 can be determined by detecting the labeled compound in a complex. For example, targets can be labeled with ¹²⁵I, ³⁵S, ¹⁴C, or ³H, either directly or indirectly, and the radioisotope detected by direct counting of radioemmission or by scintillation counting. Alternatively, compounds can be labeled, e.g., with, for example, horseradish peroxidase, alkaline phosphatase, or luciferase, and the enzymatic label detected by determination of conversion of an appropriate substrate to product.

It is also within the scope of this invention to determine the ability of a compound to interact with XBP-1 and/or IRE-1 without the labeling of any of the interactants. For example, a microphysiometer can be used to detect the interaction of a compound with XBP-1 without the labeling of either the compound or the XBP-1 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and XBP-1.

Exemplary target molecules of XBP-1 include: XBP-1-responsive elements, for example, upstream regulatory regions from genes such as Dgat2, Scd1, and Acc2, PCSK9, or α-1 antitrypsin, α-fetoprotein. Other examples can include regulatory regions of the chaperone genes such as members of the family of Glucose Regulated Proteins (GRPs) such as GRP78 (BiP) and GRP94 (endoplasmin), as well as other chaperones such as calreticulin, protein disulfide isomerase, Erdj4, EDEM and ERp72. XBP-1 targets are taught, e.g. In Clauss et al, Nucleic Acids Research 1996. 24:1855 also include CRE and TRE sequences.

In another embodiment, a different (i.e., non-XBP-1) molecule acting in a pathway involving XBP-1 that acts upstream (e.g., IRE-1) or downstream (e.g., ATF6α or cochaperone proteins that activate ER resident HspTO proteins, such as p58IPK) of XBP-1 can be included in an indicator composition for use in a screening assay. Compounds identified in a screening assay employing such a molecule would also be useful in modulating XBP-1 activity, albeit indirectly. IRE-1 is one exemplary IRE-1 substrate (e.g., the autophosphorylation of IRE-1). In another embodiment, the endoribonuclease activity of IRE-1 can be measured, e.g., by detecting the splicing of XBP-1 using techniques that are known in the art. The activity of IRE-1 can also be measured by measuring the modulation of biological activity associated with XBP-1.

In another embodiment, a different (i.e., non-XBP-1) molecule acting in a pathway involving XBP-1 that acts upstream (e.g., IRE-1) or downstream (e.g., ATF6α or cochaperone proteins that activate ER resident HspTO proteins, such as p58^{IPK}) of XBP-1 can be included in an indicator composition for use in a screening assay.

The cells of the invention can express endogenous XBP-1 and/or IRE-1, or can be engineered to do so. For example, a cell that has been engineered to express the XBP-1 protein and/or a non XBP-1 protein can be produced by introducing into the cell an expression vector encoding the protein.

In one embodiment, to specifically assess the role of agents that modulate the expression and/or activity of unspliced or spliced XBP-1 protein, retroviral gene transduction of cells deficient in XBP-1 with spliced XBP-1 or a form of XBP-1 which cannot be spliced can be performed. For example, a construct in which mutations at in the loop structure of XBP-1 (e.g., positions −1 and +3 in the loop structure of XBP-1) can be generated. Expression of this construct in cells results in production of the unspliced form of XBP-1 only. Using such constructs, the ability of a compound to modulate a particular form of XBP-1 can be detected. In one embodiment, a compound modulates one form of XBP-1, e.g., spliced XBP-1, without modulating the other form, e.g., unspliced XBP-1.

Recombinant expression vectors that can be used for expression of XBP-1, IRE-1, or a molecule in a signal transduction pathway involving XBP-1 (e.g., a protein which acts upstream or downstream of XBP-1) or a molecule in a signal transduction pathway involving XBP-1 in the indicator cell are known in the art. For example, the XBP-1 cDNA is first introduced into a recombinant expression vector using standard molecular biology techniques. A cDNA can be obtained, for example, by amplification using the polymerase chain reaction (PCR) or by screening an appropriate cDNA library. The nucleotide sequences of cDNAs for XBP-1 or a molecule in a signal transduction pathway involving XBP-1 (e.g., human, murine and yeast) are known in the art and can be used for the design of PCR primers that allow for amplification of a cDNA by standard PCR methods or for the design of a hybridization probe that can be used to screen a cDNA library using standard hybridization methods.

Following isolation or amplification of a cDNA molecule encoding, for example, XBP-1, the DNA fragment is introduced into an expression vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “host cell” is intended to refer to a cell into which a nucleic acid molecule of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Preferably a host cell is a mammalian cell, e.g., a human cell. In particularly preferred embodiments, it is a epithelial cell.

As used herein, the term “transgenic cell” refers to a cell containing a transgene.

As used herein, a “transgenic animal” includes an animal, e.g., a non-human mammal, e.g., a swine, a monkey, a goat, or a rodent, e.g., a mouse, in which one or more, and preferably essentially all, of the cells of the animal include a transgene. The transgene is introduced into the cell, directly or indirectly by introduction into a precursor of the cell, e.g., by microinjection, transfection or infection, e.g., by infection with a recombinant virus. The term genetic manipulation includes the introduction of a recombinant DNA molecule. This molecule may be integrated within a chromosome, or it may be extrachromosomally replicating DNA.

The recombinant expression vectors of the invention comprise a nucleic acid molecule in a form suitable for expression of the nucleic acid molecule in a host cell, which means that the recombinant expression vectors include one or more regulatory sequences, selected on the basis of the host cells to be used for expression and the level of expression desired, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in. Enzymology 185, Academic Press, San Diego, Calif. (1990). Regulatory sequences include those which direct constitutive expression of a nucleotide sequence in many types of host cell, those which direct expression of the nucleotide sequence only in certain host cells (e.g., tissue-specific regulatory sequences) or those which direct expression of the nucleotide sequence only under certain conditions (e.g., inducible regulatory sequences).

When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987) Nature 329:840) and pMT2PC (Kaufman et al. (1987), EMBO J. 6:187-195). A variety of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp 167-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Vector DNA can be introduced into mammalian cells via conventional transfection techniques. As used herein, the various forms of the term “transfection” are intended to refer to a variety of art-recognized techniques for introducing foreign nucleic acid (e.g., DNA) into mammalian host cells, including calcium phosphate co-precipitation, DEAE-dextran-mediated transfection, lipofection, or electroporation. Suitable methods for transfecting host cells can be found in Sambrook et al. (Molecular Cloning: A Laboratory Manual, 2nd Edition, Cold. Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on a separate vector from that encoding XBP-1 or, more preferably, on the same vector. Cells stably transfected with the introduced nucleic acid can be identified by drug selection (e.g., cells that have incorporated the selectable marker gene will survive, while the other cells die).

In one embodiment, within the expression vector coding sequences are operatively linked to regulatory sequences that allow for constitutive expression of the molecule in the indicator cell (e.g., viral regulatory sequences, such as a cytomegalovirus promoter/enhancer, can be used). Use of a recombinant expression vector that allows for constitutive expression of, for example, XBP-1 and/or IRE-1 in the indicator cell is preferred for identification of compounds that enhance or inhibit the activity of the molecule. In an alternative embodiment, within the expression vector the coding sequences are operatively linked to regulatory sequences of the endogenous gene for XBP-1 (i.e., the promoter regulatory region derived from the endogenous gene). Use of a recombinant expression vector in which expression is controlled by the endogenous regulatory sequences is preferred for identification of compounds that enhance or inhibit the transcriptional expression of the molecule.

C. Cell-Free Assays

In another embodiment, the indicator composition is a cell free composition. XBP-1 and/or IRE-1 protein expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

In one embodiment, compounds that specifically modulate XBP-1 and/or IRE-1 activity are identified based on their ability to modulate the interaction of XBP-1 and/or IRE-1 with a target molecule to which XBP-1 binds. The target molecule can be a DNA molecule, e.g., an XBP-1-responsive element, such as the regulatory region of a chaperone gene, lipogenic gene) or a protein molecule. Suitable assays are known in the art that allow for the detection of protein-protein interactions (e.g., immunoprecipitations, two-hybrid assays and the like) or that allow for the detection of interactions between a DNA binding protein with a target DNA sequence (e.g., electrophoretic mobility shift assays, DNAse I footprinting assays, chromatin immunoprecipitations assays and the like). By performing such assays in the presence and absence of test compounds, these assays can be used to identify compounds that modulate (e.g., inhibit or enhance) the interaction of XBP-1 and/or IRE-1 with a target molecule.

In one embodiment, the amount of binding of XBP-1 and/or IRE-1 to the target molecule in the presence of the test compound is greater than the amount of binding of XBP-1 and/or IRE-1 to the target molecule in the absence of the test compound, in which case the test compound is identified as a compound that enhances binding of XBP-1 (or IRE-1) to a target. In another embodiment, the amount of binding of the XBP-1 (or IRE-1) to the target molecule in the presence of the test compound is less than the amount of binding of the XBP-1 (or IRE-1) to the target molecule in the absence of the test compound, in which case the test compound is identified as a compound that inhibits binding of XBP-1 (or IRE-1) to the target.

Binding of the test compound to XBP-1 and/or IRE-1 can be determined either directly or indirectly as described above. Determining the ability of XBP-1(or IRE-1) protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In the methods of the invention for identifying test compounds that modulate an interaction between XBP-1 (or IRE-1) protein and a target molecule, the complete XBP-1 (or IRE-1) protein can be used in the method, or, alternatively, only portions of the protein can be used. For example, an isolated XBP-1b-ZIP structure (or a larger subregion of XBP-1 that includes the b-ZIP structure) can be used. In another example, a form of XBP-1 comprising the splice junction can be used (e.g., a portion including from about nucleotide 506 to about nucleotide 532). The degree of interaction between the protein and the target molecule can be determined, for example, by labeling one of the proteins with a detectable substance (e.g., a radiolabel), isolating the non-labeled protein and quantitating the amount of detectable substance that has become associated with the non-labeled protein. The assay can be used to identify test compounds that either stimulate or inhibit the interaction between the XBP-1(or IRE-1) protein and a target molecule. A test compound that stimulates the interaction between the protein and a target molecule is identified based upon its ability to increase the degree of interaction between, e.g., spliced XBP-1 and a target molecule as compared to the degree of interaction in the absence of the test compound and such a compound would be expected to increase the activity of spliced XBP-1 in the cell. A test compound that inhibits the interaction between the protein and a target molecule is identified based upon its ability to decrease the degree of interaction between the protein and a target molecule as compared to the degree of interaction in the absence of the compound and such a compound would be expected to decrease spliced XBP-1 activity.

In one embodiment of the above assay methods of the present invention, it may be desirable to immobilize eitherXBP-1 (and/or IRE-1) or a respective target molecule for example, to facilitate separation of complexed from uncomplexed forms of one or both of the proteins, or to accommodate automation of the assay. Binding of a test compound to, for example, an XBP-1 protein, or interaction of an XBP-1 protein with a target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided in which a domain that allows one or both of the proteins to be bound to a matrix is added to one or more of the molecules. For example, glutathione-S-transferase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or XBP-1 (or IRE-1) protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, either an XBP-1 protein or a molecule in a signal transduction pathway involving XBP-1, or a target molecule can be immobilized utilizing conjugation of biotin and streptavidin. Biotinylated protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with protein or target molecules but which do not interfere with binding of the protein to its target molecule can be derivatized to the wells of the plate, and unbound target or XBP-1 (or IRE-1) protein is trapped in the wells by antibody conjugation. Methods for detecting such complexes, in addition to those described above for the GST-immobilized complexes, include immunodetection of complexes using antibodies reactive with XBP-1 or a molecule in a signal transduction pathway involving XBP-1 or target molecule, as well as enzyme-linked assays which rely on detecting an enzymatic activity associated with the XBP-1 or IRE-1, protein or target molecule.

In yet another aspect of the invention, the XBP-1 protein (or IRE-1) or fragments thereof can be used as “bait proteins” e.g., in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent W094/10300), to identify other proteins, which bind to or interact with XBP-1 (“binding proteins” or “bp”) and are involved in XBP-1 activity. Such XBP-1-binding proteins are also likely to be involved in the propagation of signals by the XBP-1 proteins or XBP-1 targets such as, for example, downstream elements of an XBP-1-mediated signaling pathway. Alternatively, such XBP-1-binding proteins can be XBP-1 inhibitors.

The two-hybrid system is based on the modular nature of most transcription factors, which consist of separable DNA-binding and activation domains. Briefly, the assay utilizes two different DNA constructs. In one construct, the gene that codes for an XBP-1 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an XBP-1 dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the XBP-1 protein or a molecule in a signal transduction pathway involving XBP-1.

C. Assays Using Knock-Down or Knock-Out Cells

In another embodiment, the invention provides methods for identifying compounds that modulate a biological effect of XBP-1 or a molecule in a signal transduction pathway involving XBP-1 using cells deficient in XBP-1 (or e.g., IRE-1). Cells deficient in XBP-1 or a molecule in a signal transduction pathway involving XBP-1 or in which XBP-1 or a molecule in a signal transduction pathway involving XBP-1 is knocked down can be used to identify agents that modulate a biological response regulated by XBP-1 by means other than modulating XBP-1 itself (i.e., compounds that “rescue” the XBP-1 deficient phenotype). Alternatively, a “conditional knock-out” system, in which the gene is rendered non-functional in a conditional manner, can be used to create deficient cells for use in screening assays. For example, a tetracycline-regulated system for conditional disruption of a gene as described in WO 94/29442 and U.S. Pat. No. 5,650,298 can be used to create cells, or animals from which cells can be isolated, be rendered deficient in XBP-1 (or a molecule in a signal transduction pathway involving XBP-1 e.g., IRE-1) in a controlled manner through modulation of the tetracycline concentration in contact with the cells.

In the screening method, cells deficient in XBP-1 or a molecule in a signal transduction pathway involving XBP-1 can be contacted with a test compound and a biological response regulated by XBP-1 or a molecule in a signal transduction pathway involving XBP-1 can be monitored. Modulation of the response in cells deficient in XBP-1 or a molecule in a signal transduction pathway involving XBP-1 (as compared to an appropriate control such as, for example, untreated cells or cells treated with a control agent) identifies a test compound as a modulator of the XBP-1(or e.g., IRE-1) regulated response. In another embodiment, to specifically assess the role of agents that modulate unspliced or spliced XBP-1 protein, retroviral gene transduction of cells deficient in XBP-1, to express spliced XBP-1 or a form of XBP-1 which cannot be spliced can be performed. For example, a construct in which mutations at in the loop structure of XBP-1 (e.g., positions −1 and +3 in the loop structure of XBP-1) can be generated. Expression of this construct in cells results in production of the unspliced form of XBP-1 only. Using such constructs, the ability of a compound to modulate a particular form of XBP-1 can be detected. For example, in one embodiment, a compound modulates one form of XBP-1 without modulating the other form.

In one embodiment, the test compound is administered directly to a non-human knock out animal, preferably a mouse (e.g., a mouse in which the XBP gene or a gene in a signal transduction pathway involving XBP-1 is conditionally disrupted by means described above, or a chimeric mouse in which the lymphoid organs are deficient in XBP-1 as described above), to identify a test compound that modulates the in vivo responses of cells deficient in XBP-1(or e.g., IRE-1). In another embodiment, cells deficient in XBP-1 (or e.g., IRE-1) are isolated from the non-human XBP-1 or a molecule in a signal transduction pathway involving an XBP-1 deficient animal, and contacted with the test compound ex vivo to identify a test compound that modulates a response regulated by XBP-1(or e.g., IRE-1) in the cells.

Cells deficient in XBP-1 or a molecule in a signal transduction pathway involving XBP-1 can be obtained from non-human animals created to be deficient in XBP-1 or a molecule in a signal transduction pathway involving XBP-1 Preferred non-human animals include monkeys, dogs, cats, mice, rats, cows, horses, goats and sheep. In preferred embodiments, the deficient animal is a mouse. Mice deficient in XBP-1 or a molecule in a signal transduction pathway involving XBP-1 can be made using methods known in the art. Non-human animals deficient in a particular gene product typically are created by homologous recombination. Briefly, a vector is prepared which contains at least a portion of the gene into which a deletion, addition or substitution has been introduced to thereby alter, e.g., functionally disrupt, the endogenous XBP-1 (or e.g., IRE-1 gene). The gene preferably is a mouse gene. For example, a mouse XBP-1 gene can be isolated from a mouse genomic DNA library using the mouse XBP-1 cDNA as a probe. The mouse XBP-1 gene then can be used to construct a homologous recombination vector suitable for modulating an endogenous XBP-1 gene in the mouse genome. In a preferred embodiment, the vector is designed such that, upon homologous recombination, the endogenous gene is functionally disrupted (i.e., no longer encodes a functional protein; also referred to as a “knock out” vector).

Alternatively, the vector can be designed such that, upon homologous recombination, the endogenous gene is mutated or otherwise altered but still encodes functional protein (e.g., the upstream regulatory region can be altered to thereby alter the expression of the endogenous XBP-1 protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced gene has homologously recombined with the endogenous gene are selected (see e.g., Li, E. et al. (1992) Cell 69:915). The selected cells are then injected into a blastocyst of an animal (e.g., a mouse) to form aggregation chimeras (see e.g., Bradley, A. In Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, retroviral transduction of donor bone marrow cells from both wild type and null mice can be performed, e.g., with the XBP-1 unspliced, DN or spliced constructs to reconstitute irradiated RAG recipients. This will result in the production of mice whose lymphoid cells express only unspliced, or only spliced XBP-1 protein, or which express a dominant negative version of XBP-1. Cells from these mice can then be tested for compounds that modulate a biological response regulated by XBP-1.

In another embodiment, XBP-1 may be temporally deleted to, for example, circumvent embryonic lethality.

In another embodiment, a molecule which mediates RNAi, e.g., double stranded RNA can be used to knock down expression of XBP-1 or a molecule in a signal transduction pathway involving XBP-1. For example, an XBP-1-specific RNAi vector has been constructed by inserting two complementary oligonucleotides 5′-GGGATTCATGAATGGCCCTTA-3′ (SEQ ID NO.:24) into the pBS/U6 vector as described (Sui et al. 2002 Proc Natl Acad Sci USA 99: 5515-5520).

In one embodiment of the screening assay, compounds tested for their ability to modulate a biological response regulated by XBP-1 or a molecule in a signal transduction pathway involving XBP-1 are contacted with deficient cells by administering the test compound to a non-human deficient animal in vivo and evaluating the effect of the test compound on the response in the animal.

The test compound can be administered to a non-knock out animal as a pharmaceutical composition. Such compositions typically comprise the test compound and a pharmaceutically acceptable carrier. As used herein the term “pharmaceutically acceptable carrier” includes any and all solvents, dispersion media, coatings, antibacterial and antifungal compounds, isotonic and absorption delaying compounds, and the like, compatible with pharmaceutical administration. The use of such media and compounds for pharmaceutically active substances is well known in the art. Except insofar as any conventional media or compound is incompatible with the active compound, use thereof in the compositions is contemplated. Supplementary active compounds can also be incorporated into the compositions. Pharmaceutical compositions are described in more detail below.

In another embodiment, compounds that modulate a biological response regulated by XBP-1 or a signal transduction pathway involving XBP-1 are identified by contacting cells deficient in XBP-1 ex vivo with one or more test compounds, and determining the effect of the test compound on a read-out. In one embodiment, XBP-1 deficient cells contacted with a test compound ex vivo can be re-administered to a subject.

For practicing the screening method ex vivo, cells deficient, e.g., in XBP-1, IRE-1, can be isolated from a non-human XBP-1, IRE-1, deficient animal or embryo by standard methods and incubated (i.e., cultured) in vitro with a test compound. Cells (e.g., B cells, hepatocytes, MEFs) can be isolated from e.g., XBP-1, IRE-1 deficient animals by standard techniques.

In another embodiment, cells deficient in more than one member of a signal transduction pathway involving XBP-1 can be used in the subject assays.

Following contact of the deficient cells with a test compound (either ex vivo or in vivo), the effect of the test compound on the biological response regulated by XBP-1 or a molecule in a signal transduction pathway involving XBP-1 can be determined by any one of a variety of suitable methods, such as those set forth herein, e.g., including light microscopic analysis of the cells, histochemical analysis of the cells, production of proteins, induction of certain genes, e.g., chaperone genes, lipogenic genes.

D. Test Compounds

A variety of test compounds can be evaluated using the screening assays described herein. The term “test compound” includes any reagent or test agent which is employed in the assays of the invention and assayed for its ability to influence the expression and/or activity of XBP-1 and/or IRE-1. More than one compound, e.g., a plurality of compounds, can be tested at the same time for their ability to modulate the expression and/or activity of, e.g., XBP-1, in a screening assay. The term “screening assay” preferably refers to assays which test the ability of a plurality of compounds to influence the readout of choice rather than to tests which test the ability of one compound to influence a readout. Preferably, the subject assays identify compounds not previously known to have the effect that is being screened for. In one embodiment, high throughput screening can be used to assay for the activity of a compound.

In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909) peptoids (Zuckermann. (1994). J. Med. Chem. 37:2678) oligocarbamates (Cho et al. (1993). Science. 261:1303), and hydantoins (DeWitt et al. supra). An approach for the synthesis of molecular libraries of small organic molecules with a diversity of 104-105 as been described (Carell et al. (1994). Angew. Chem. Int. Ed. Engl. 33:2059-; Carell et al. (1994) Angew. Chem. Int. Ed. Engl. 33:2061-).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422; Horwell et al. (1996) Immunopharmacology 33:68-; and in Gallop el al. (1994); J. Med. Chem. 37:1233-.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310). In still another embodiment, the combinatorial polypeptides are produced from a cDNA library.

Exemplary compounds which can be screened for activity include, but are not limited to, peptides, nucleic acids, carbohydrates, small organic molecules, and natural product extract libraries.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), and 6) mutant forms of XBP-1 (or e.g., IRE-1 molecules, e.g., dominant negative mutant forms of the molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Nail Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) Mol. Biol. 222:301-310; Ladner supra.).

Compounds identified in the subject screening assays can be used in methods of modulating one or more of the biological responses regulated by XBP-1. It will be understood that it may be desirable to formulate such compound(s) as pharmaceutical compositions (described supra) prior to contacting them with cells.

Once a test compound is identified that directly or indirectly modulates, e.g., XBP-1 expression or activity, by one of the variety of methods described hereinbefore, the selected test compound (or “compound of interest”) can then be further evaluated for its effect on cells, for example by contacting the compound of interest with cells either in vivo (e.g., by administering the compound of interest to a subject) or ex vivo (e.g., by isolating cells from the subject and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

E. Computer Assisted Design of Modulators of XBP-1 and/or IRE-1

Computer-based analysis of a protein with a known structure can also be used to identify molecules which will bind to the protein. Such methods rank molecules based on their shape complementary to a receptor site. For example, using a 3-D database, a program such as DOCK can be used to identify molecules which will bind to XBP-1 or a molecule in a signal transduction pathway involving XBP-1. See DesJarlias et al. (1988) J. Med. Chem. 31:722; Meng et al. (1992) J. Computer Chem. 13:505; Meng et al. (1993) Proteins 17:266; Shoichet et al. (1993) Science 259:1445. In addition, the electronic complementarity of a molecule to a targeted protein can also be analyzed to identify molecules which bind to the target. This can be determined using, for example, a molecular mechanics force field as described in Meng et al. (1992) J. Computer Chem. 13:505 and Meng et al. (1993) Proteins 17:266. Other programs which can be used include CLIX which uses a GRID force field in docking of putative ligands. See Lawrence et al. (1992) Proteins 12:31; Goodford et al. (1985) J. Med. Chem. 28:849; Boobbyer et al. (1989) J. Med. Chem. 32:1083.

The instant invention also pertains to compounds identified in the subject screening assays.

### V. Kits of the Invention

Another aspect of the invention pertains to kits for carrying out the screening assays or modulatory methods of the invention. For example, a kit for carrying out a screening assay of the invention can include an indicator composition comprising XBP-1 and/or IRE-1, means for measuring a readout (e.g., protein secretion) and instructions for using the kit to identify modulators of biological effects of XBP-1. In another embodiment, a kit for carrying out a screening assay of the invention can include cells deficient in XBP-1 or a molecule in a signal transduction pathway involving XBP-1, means for measuring the readout and instructions for using the kit to identify modulators of a biological effect of XBP-1.

In another embodiment, the invention provides a kit for carrying out a modulatory method of the invention. The kit can include, for example, a modulatory agent of the invention (e.g., XBP-1 inhibitory or stimulatory agent) in a suitable carrier and packaged in a suitable container with instructions for use of the modulator to modulate a biological effect of XBP-1.

This invention is further illustrated by the following examples which should not be construed as limiting. The contents of all references, patents and published patent applications cited throughout this application are hereby incorporated by reference.

### EXAMPLES

The following materials and methods were used in the Examples.

### Reagents

LPS, Pam3CSK4, the diacylated synthetic lipoprotein FSL1 and other TLR ligands were purchased from Invivogen. Muramyl dipeptide, tunicamycin, sulfasalazine, SB203580, SP600125 PD98059, diphenyleneiodonium chloride and apocynin were purchased from Calbiochem.

### Immunoblotting

Total cell extracts were loaded on SDS-PAGE for separation and transferred to PDVF membranes. For Western blot the following antibodies were used: anti-XBP-1s (Biolegend), anti-phospho PERK, IRE1 and phospho-JNK (Cell Signaling), ATF4, HSP90, IkappaBβ and CHOP (Santa Cruz Biotechnology), anti-ATF6α and anti proIL-1β.

### RNA Extraction, RT-PCR and XBP-1 Splicing Assay

Total RNA was prepared using Trizol (Invitrogen) and cDNA synthesis with High capacity cDNA Reverse transcription kit purchased from Applied Biosystems. Quantitative RT-PCR was performed employing SYBR green fluorescent reagent on a Mx3005P® QPCR System from Strategene. The relative amounts of mRNA were calculated from the values of comparative threshold cycle by using β-actin as control.

The following primers were used:

for mIL-1β
FW: 5′-CAACCAACAAGTGATATTCTCCATG,
RW 5′-GATCCACACTCTCCAGCTGCA;
mIL-6,
FW 5′-GAGGATACCACTCCCAACAGACC,
RW 5′-AAGTGCATCATCGTTGTTCATACA;
mISG15,
FW 5′-AGCGGAACAAGTCACGAAGAC,
RW 5′-TGGGGCTTTAGGCCATACTC;
mTNF,
FW 5′-CATCTTCTCAAAATTCGAGTGACAA,
RW 5′-TGGGAGTAGACAAGGTACAACCC;
mActin,
FW: 5′TACCACCATGTACCCAGGCA,
RW 5′CTCAGGAGGAGCAATGATCTTGAT;
Bip,
FW 5′-TCATCGGACGCACTTGGA,
RW 5′-CAACCACCTTGAATGGCAAGA
CHOP
FW 5′-GTCCCTAGCTTGGCTGACAGA
RW 5′-TGGAGAGCGAGGGCTTTG;
PDI,
FW 5′-CAAGATCAAGCCCCACCTGAT
RW AGTTCGCCCCAACCAGTACTT;
ERDJ4,
FW 5′-CCCCAGTGTCAAACTGTACCAG
RW 5′-AGCGTTTCCAATTTTCCATAAATT;
WSF1,
FW 5′-CCATCAACATGCTCCCGTTC
RW 5′-GGGTAGGCCTCGCCAT.

XBP-1 splicing assay was performed as previously described (N. N. Iwakoshi et al., Nat Immunol 4, 321 (Apr. 1, 2003)). In brief, PCR primers 5′-ACACGCTTGGGAATGGACAC-3′ and 5′-CCATGGGAAGATGTTCTGGG-3′ encompassing the spliced sequences in XBP-1 mRNA were used for the PCR amplification, the PCR products subjected to electrophoresis on a 2.5% agarose gel and visualized by ethidium bromide staining.

### ELISA

ELISA for mouse IL-6 was done by using 4 μg/ml of capture antibody, 2 μg/ml of biotinylated secondary antibody (BD Biosciences) and a 1:1000 dilution of alkaline phosphatase-conjugated streptavidin (Sigma).

### Knockdown of IRE1α, TRAF6 and NEMO

Stable J744 macrophage populations were generated by targeting IRE1α, TRAF6 and NEMO mRNA by using the lentiviral delivery of specific shRNAs followed by puromycin (4 μg/ml) selection. shRNA targeting luciferase mRNA were used as a control. For every gene five different targeting constructs were screened and the most efficients were selected. Optimal targeting sequences identified for mouse IRE1, TRAF6 and NEMO are 5′-GCTCGTGAATTGATAGAGAAA; 5′-CCCAGGCTGTTCATAATGTTA and 5′-AGACTACGACAGCCACATTAA, respectively.

Transduction of J774 Cells with Active IRE1.

J774 macrophages were transduced as described (J. Summerton, Ann NY Acad Sci 1058, 62 (Nov. 1, 2005)). Briefly the cells were incubated for 1 h with 6 μg/ml of endo-porter (GeneTools) and various amounts of an active recombinant IRE1 fragment spanning mainly the kinase and ribonuclease domains of IRE1.

### Mice

MyD88 deficient mice are on a C57BL/6 background. C3H/HeOuJ, C3H/HeJ, Lps2 (TRIF deficient) and TLR2 deficient mice were purchased from Jackson Laboratories. Mice lacking XBP-1 in hematopoietic cells including macrophages were generated from mating XBP-1 flox mice harboring loxP sites in the first and second intron of the XBP-1 gene to mice expressing an interferon-dependent cre recombinase as previously described (A. Lee, E. F. Scapa, D. E. Cohen, L. H. Glimcher, Science 320, 1492 (Jun. 13, 2008)). Five week old mice were intraperitoneally injected 1 or 3 times with 250 μg of poly(I:C) at 2 day intervals to induce cre expression and used for experiments 2-3 weeks to several months after the final poly(I:C) injection. Sex-matched XBP-1flox littermates injected with poly(I:C) were used as WT controls throughout the study.

### Bone Marrow-Derived Macrophages (BMM)

Bone marrow was collected from femurs in DMEM/F12 medium supplemented with 10% L-929 cell-conditioned media, 10% heat inactivated FCS and 1 ng/ml IL-3 (PreproTech) and antibiotics. After two days culture non-adherent precursors were plated in 12 wells plate at 2×105 cells/well in DMEM/F12 medium supplemented with 10% L-929 cells conditioned media, 10% heat inactivated FCS and antibiotics for 6 to 10 days. The media was changed every other day. Except for Francisella infection, all experiments involving stimulation kinetics were performed to harvest all samples simultaneously.

Francisella tularensis LVS Infection

F. tularensis subsp. holarctica Live Vaccine Strain (LVS) was obtained from the New England Regional Center of Excellence/Biodefense and Emerging Infectious Diseases (Boston, Mass.). LVS was grown in modified Mueller-Hinton broth, harvested and frozen at −80 oC in 1 ml aliquots (1010 CFU/ml). For infection 1 ml LVS stock is grown in 25 ml of Mueller-Hinton broth at 37 oC, 250 rpm, overnight to OD600 of 0.3-0.4 Antibiotic-free macrophages were infected with F. tularensis LVS at a multiplicity of infection (MOI) of 10:1 (bacterium-to-macrophage). 2 h after infection, the cell monolayer was washed three times with sterile PBS and incubated for 45 min with media containing 50 μg/ml gentamicin to eliminate extracellular bacteria. Macrophages were then washed three times with PBS and gentamicin-free media and analyzed as described. For aerosol infection, 1 ml LVS stock was grown in 25 ml of Mueller-Hinton broth at 37 oC, 250 rpm, overnight. The inoculation stock is diluted to 7×107 CFU/ml in sterile PBS containing 20% glycerol. LVS aerosols are generated with a Lovelace nebulizer using a commercial nose-only exposure apparatus (InTox Products, Moriarty, N. Mex.). In each experiment, mice were exposed to LVS containing aerosols for 30 minutes, followed by 10 minutes of clean air, where aerosols are delivered to the exposure ports at a flow rate of 6 l/min. Seven days after aerosol infection, lungs, liver and spleen from infected mice were homogenized in 10 ml of sterile PBS and the suspension plated on Mueller-Hinton plates to determine the bacterial burden.

### Example 1

### TLRs Repress a Classic ER Stress Response Pathway in Macrophages

The ability of TLRs to activate the three branches of the ER stress response was tested. J774 macrophages were stimulated with the TLR4 and TLR2 agonists, LPS and Pam3CSK4, and IRE1α, PERK and ATF6 activation were analyzed. ATF6 activation was tested by monitoring the liberation of its cleaved fragment, and PERK and IRE1α activation by examining their phosphorylation status. Clear activation of IRE1α was evident by appearance of the slower migrating phosphorylated species. However, neither activation of ATF6 or PERK nor induction of the classic ER stress downstream target genes, CHOP and BiP, was observed. Surprisingly, despite activating IRE1, LPS did not induce the expression of the XBP-1 target genes Erdj4 or PDI. To further examine the effects of TLR signaling on the classical. ER stress response, cells were stimulated with tunicamycin (TM), a potent pharmacologic ER-stress inducer, in the presence or absence of LPS. TM alone induced activation of all three branches of the ER stress response as expected. As above, it was found that LPS did not induce a classic ER-stress response as measured by CHOP, BiP, Erdj4 and PDI expression. In fact, LPS co-treatment dramatically repressed the induction of mRNAs encoding these ER-stress induced genes including CHOP, PDI and Erdj4 and the proteolytic processing of ATF6α by TM in J774 macrophages. By analyzing primary macrophages from MyD88 deficient mice, it was observed that TLR2 dampening of the ER-stress response was MyD88 dependent (Pam3CSK4) while TLR4 dampening was only partially dependent on MyD88. Therefore, TLRs trigger a biased ER-stress response by activating IRE1 while inhibiting the PERK and ATF6 pathways.

### Example 2

### TLRs Trigger XBP-1 Activation in Macrophages

Since TLR signaling did not induce the expression of classic XBP-1 target genes, it was determined whether TLRs activated XBP-1 or whether IRE1 activation by TLR signaling engaged a novel XBP-1 independent pathway. IRE1-mediated splicing of XBP-1 mRNA generates XBP-1 s, a potent transcriptional activator. To interrogate whether bacterial products that act as TLR agonists can promote XBP-1 activation, J774 macrophages were stimulated with the TLR4 agonist LPS. Splicing of XBP-1 mRNA and production of XBP-1s protein was detected as early as 3 hours after LPS stimulation. As above, CHOP and ATF6α, two transcription factors that are induced or activated upon ER-stress were not detected following TLR stimulation. Interrogation of a panel of TLR agonists revealed that agonists engaging TLR2, TLR4 and TLR5 but not TLR3, TLR7 or TLR9 promoted XBP-1 splicing. XBP-1s production upon LPS stimulation was IRE1α dependent as demonstrated by defective LPS induced XBP-1 mRNA splicing in IRE1α shRNA knockdown cells. TLR activation of XBP-1 was also observed in primary macrophages from wild type (WT) mice upon LPS stimulation. Importantly, infection of WT primary macrophages with three different pathogens, Mycobacterium tuberculosis, Listeria monocytogenes and Franciscella tularensis induced XBP-1 spliced mRNA. Macrophages isolated from mice carrying a mutation in the signaling domain of TLR4 did not activate XBP-1 upon LPS stimulation. Similarly, the TLR2 agonists Pam3CSK4 and FSL1 did not activate XBP-1 splicing in TLR2 deficient macrophages demonstrating that XBP-1 splicing is triggered by TLRs rather than by a potential direct effect of these PAMPs on the ER.

### Example 3

### TLR Activation of XBP-1 is TRAF6 and NADPH Oxidase Dependent

To identify the downstream effectors of TLRs that mediate XBP-1 activation, XBP-1 splicing in macrophages from mice deficient for the TIR adaptors MyD88, TRIF, or TIRAP was interrogated. TLR2 induced splicing of XBP-1 was dependent on the TIRAP-MyD88 signaling pathway, while TRIF and MyD88 deficiency only partially inhibited TLR4 activation of XBP-1, indicating that both MyD88 dependent and MyD88 independent (TRIF dependent) signaling lead to TLR4-induced XBP-1 splicing. Both TRIF and MyD88 are known to engage the signaling molecule TRAF6 and the downstream NF-κB scaffolding protein NEMO (T. Kawai, S. Akira, Semin Immunol 19, 24 (Feb. 1, 2007)). Analysis of J774 macrophage populations stably expressing shRNAs targeting TRAF6 or NEMO revealed that TRAF6 was required for XBP-1 splicing; in contrast, NEMO was dispensable. As control, both NEMO and TRAF6 knockdown populations failed to induce IL-6 transcription upon LPS stimulation. Further, pharmacological inhibition of NF-κB, p38, JNK and MEK pathways did not affect LPS-induced XBP-1 splicing confirming that splicing is mediated by TRAF6 activation independently of NF-κB, p38 and JNK pathways.

TRAF proteins can bind and activate nicotinamide adenine dinucleotide phosphate (NADPH)-oxidase, a membrane-bound enzyme complex found in the plasma membrane as well as in phagosome membranes (Matsuzawa et al., Nat Immunol 6, 587 (Jun. 1, 2005); Takeshita et al., Eur. J. Immunol. 35, 2477 (Aug. 1, 2005);Y. J. Ha, J. R. Lee, J Immunol 172, 231 (Jan. 1, 2004)). NADPH oxidases are a significant source of oxidative stress by producing reactive oxygen species (ROS). Oxidative stress and ER-stress are closely linked pathways—while ER-stress is a well-known amplifier of oxidative stress, ROS lead to ER-stress as part of an adaptive mechanism to preserve cell function and survival (Yokouchi et al., Journal of Biological Chemistry 283, 4252 (Feb. 15, 2008); J. D. Malhotra, R. J. Kaufman, Antioxid Redox Signal 9, 2277 (Dec. 1, 2007)). The NADPH-oxidase complex mediates the induction of reactive oxygen species (ROS) by TLRs in macrophages (N. Grandvaux, A. Soucy-Faulkner, K. Fink, Biochimie 89, 1113 (Sep. 1, 2007)). Therefore, it was investigated if LPS-mediated XBP-1 splicing was dependent on NADPH oxidase. Use of NADPH oxidase inhibitors diphenyleneiodonium chloride (DPI) or apocynin abolished XBP-1 splicing by LPS, but not by TM treatment demonstrating that TLRs mediate XBP-1 splicing by activating the NADPH-oxidase complex.

### Example 4

### TLR Activation of XBP-1 Regulates Cytokine Production by Macrophages

To establish the function of XBP-1 in TLR signaling, XBP-1 flox/floxMxCre mice (hereafter called XBP-1Δ) lacking XBP-1 in macrophages and other hematopoietic lineage cells was used (A. Lee, E. F. Scapa, D. E. Cohen, L. H. Glimcher, Science 320, 1492 (Jun. 13, 2008)). Consistent with the results above, no induction of classic ER-stress related genes such as CHOP or PDI was observed upon stimulation of XBP-1Δ macrophages or WT macrophages with LPS. As control, TM induction of PDI and WSF1 was reduced in XBP-1Δ macrophages indicating efficient XBP-1 deletion. However, XBP-1Δ macrophages stimulated with TLR4 or TLR2 agonists displayed impaired production secretion of IL-6 as measured by ELISA and impaired production of IL-6 TNF INFβ, ISG15 and COX2 as measured by quantitative RT-PCR. The defect was specific to certain mediators since other cytokines such as IL-1β and RANTES were unaffected by XBP-1 deficiency. Interestingly, XBP-1Δ macrophages did not display an absolute defect-rather, it was found that early (1 h) IL-6 mRNA induction was comparable to WT but that IL-6 production was defective at later (6 h) time points. Consistent with the unperturbed early cytokine profile, early induction of IκBβ degradation and JNK phosphorylation were not affected. Taken together, these findings demonstrate that XBP-1 in macrophages is required for sustained production of innate immune mediators such as IL-6 and IFNβ.

### Example 5

### XBP-1 Activation by ER Stress Enhances TLR Signaling

The experiments above suggested that activation of the IRE1-XBP-1 signaling pathway by the innate immune system in macrophages is required for optimal secretion of certain proinflammatory cytokines. Therefore, it was determined whether augmenting this pathway through pharmacologic induction of an ER stress response would enhance the natural response to microbial products. Indeed, LPS treatment increased TM-induced XBP-1 splicing but not in TLR4 unresponsive macrophages. Remarkably, the amounts of IL-6 and ISG15 produced by LPS stimulated macrophages were dramatically and synergistically increased when the cells were simultaneously ER-stressed with TM. To demonstrate that this effect was triggered by IRE1, J774 macrophages were transduced with recombinant protein encoding the cytosolic kinase and ribonuclease domain of IRE1α. A marked augmentation of IL-6 production was observed in the presence of active IRE1 and LPS. As confirmation, knockdown of IRE1α in J774 macrophages resulted in reduced IL-6 induction by TM and LPS co-treatment. Importantly, this finding was confirmed in primary WT and XBP-1Δ macrophages that were left untreated or pre-treated for 12 h with a low dose of TM and then stimulated with LPS for 3 h. In primary macrophages, WT but not XBP-1Δ macrophages displayed augmented IL-6 production.

### Example 6

### XBP-1 is Required for Immune Responses to the Intracellular Pathogen F. tularensis

XBP-1 was activated by infection of macrophages with three different pathogens that are agonists for TLR2 and/or TLR4. The physiologic relevance of these observations in the context of infection with the intracellular pathogen F. tularensis was investigated. The immune response to this intracellular bacterium and potential bioterrorism agent that causes the human disease tularemia, has been studied in mice using the attenuated live vaccine strain (LVS) (C. Rick Lyons, T. H. Wu, Ann N Y Acad Sci 1105, 238 (Jun. 1, 2007)). Optimal immunity against F. tularensis requires TLR2 (L. E. Cole et al., J Immunol 176, 6888 (Jun. 1, 2006); J. Katz, P. Zhang, M. Martin, S. N. Vogel, S. M. Michalek, Infect Immun 74, 2809 (May 1, 2006); L. E. Cole et al., Infect Immun (May 21, 2007)) and cytokine secretion including TNFα, in macrophages (K. L. Elkins, S. C. Cowley, C. M. Bosio, Ann N Y Acad Sci 1105, 284 (Jun. 1, 2007)). BMM from XBP-1Δ and WT mice were infected with the F. tularensis LVS strain and monitored production of inflammatory mediators by RT-PCR. Confirming previous results with TLR2 agonists, augmented production of IL-6, TNFα, and IL-1β in WT macrophages was found. Optimum induction of IL-6 and TNFα was XBP-1-dependent, while the increased production of IL-1β was XBP-1-independent consistent with the data with TLR agonists. TLR2 deficiency has been shown to result in decreased IL-6 and TNFα production in vivo and increased bacterial load in F. tularensis infected mice (M. Malik et al., Infect Immun 74, 3657 (Jun. 1, 2006)). TLR2-deficient macrophages failed to activate F. tularensis-induced XBP-1 splicing. To test whether loss of XBP-1 impairs the immune response to F. tularensis in vivo, WT and XBP-1Δ mice were infected with low doses of F. tularensis by aerosol exposure, a mimic of the often lethal human disease, pneumonic tularemia. Similar to TLR2-deficient mice, survival of neither XBP-1Δ nor WT mice was significantly impaired. However quantification of bacterial burden in various organs 7 days after aerosol infection revealed that bacterial burden was greater than one log higher in the spleen, lung and liver of XBP-1Δ mice compared with WT littermates. However, by day 14, both WT and. XBP-1Δ BALB/c mice had cleared the infection as determined by the absence of bacteria in the spleen and liver although XBP-1Δ lungs harbored significantly greater bacterial loads than WT lungs even at day 14. Consistent with the increased bacterial burden, histopathology of XBP-1Δ liver showed an increase in nondiscrete neutrophil-rich granulomatous lesions compared with WT liver at day 7 postinfection. Hence, XBP-1 is required for the early protective host innate immune response to infection with F. tularensis but likely is not important in the adaptive immune response to this organism. This is consistent with observations that XBP-1 splicing is not evident in T cells, and that B cells are not important in the early response to natural infection with F. tularensis (K. L. Elkins, C. M. Bosio, T. R. Rhinehart-Jones, Infect Immun 67, 6002 (Nov. 1, 1999)).

### Example 7

### The ER Stress Factor XBP-1 Regulates TLR Signaling and Innate Immunity

Certain transcription factors can modulate the amplitude and nature of innate immune responses via feedback loops that allow “fine tuning” of transcriptional programs appropriate to a given host-pathogen interaction (J. C. Roach et al., Proc Natl Acad Sci USA 104, 16245 (Oct. 9, 2007)). For example, ATF3 (M. Gilchrist et al., Nature 441, 173 (May 11, 2006)) and IRF4 (H. Negishi et al., Proc Natl Acad Sci USA 102, 15989 (Nov. 1, 2005); K. Honma et al., Proc Natl Acad Sci USA 102, 16001 (Nov. 1, 2005)) negatively regulate TLR4 signaling. Here, XBP-1 was identified as a novel regulatory factor that enhances TLR function. Several lines of evidence are provided demonstrating that XBP-1 functions in a positive feedback loop to sustain TLR signaling in macrophages. First, TLR2 and TLR4 signaling from the plasma membrane activates IRE1 to promote XBP-1 mRNA maturation and production of an active XBP-1s protein. Second, XBP-1 deficiency in macrophages impairs sustained production of specific cytokines including IL-6 and IFNβ upon stimulation with TLR agonists or infection with the intracellular pathogen F. tularensis without affecting early production of these cytokines. Third, consistent with the hypothesis that XBP-1 enhances gene transcription, pharmacological activation of XBP-1 synergistically augments TLR production of IL-6. This finding is consistent with a report showing that macrophage cell line RAW267.4 overexpressing XBP-1s or stimulated with the ER-stress inducer, thapsigargin has increased IFNβ production upon stimulation with LPS (J. Smith et al., Eur. J. Immunol. 38, 1194 (May 1, 2008)). Although it was found that XBP-1 activation by TLRs is restricted to macrophages, it is noteworthy that XBP-1 regulates IL-6 production in B-cells (N. N. Iwakoshi et al., Nat Immunol 4, 321 (Apr. 1, 2003)). Whether XBP-1 regulates TLR signaling by inhibiting a negative feedback loop or by actively enhancing gene transcription warrants further investigation. Finally, in vivo experiments demonstrate that XBP-1 is crucial for optimal resistance to aerosol infection with the human pathogen F. tularensis. Interestingly, XBP-1 is critical for survival in Caenorhabditis elegans infected with pathogenic bacteria suggesting that its role in innate immunity is evolutionarily conserved (L. Bischof et al., PLoS Pathog 4, e1000176 (Oct. 1, 2008)).

Similarities in signaling pathways stemming from TLR and ER stress receptors have been noted (K. Zhang et al., Cell 124, 587 (Feb. 10, 2006); K. Zhang, R. Kaufman, Nature 454, 455 (Jul. 24, 2008)). Both the IRE1 ER stress kinase and TLRs trigger the production of ROS and acute phase proteins and both engage NEMO and TRAF adaptors to trigger inflammatory signaling components such as NF-κB and the mitogen-activated protein kinase (MAPK) c-Jun N-terminal kinase (JNK) (T. Kawai, S. Akira, Semin Immunol 19, 24 (Feb. 1, 2007); P. Hu, Z. Han, A. D. Couvillon, R. J. Kaufman, J. H. Exton, Mol Cell Biol 26, 3071 (Apr. 1, 2006); F. Urano et al., Science 287, 664 (Jan. 28, 2000)). These similarities suggest that TLRs and ER-stressors may have co-evolved strategies to maximize the response to internal and external invaders, and are underscored by the observation that both PERK and IRE1 are evolutionarily related to proteins (PKR and RNAseL, respectively), involved in innate immunity.

As demonstrated herein, in addition to shared features in proximal signaling, TLR and IRE1/XBP-1 pathways are interconnected and cooperate to maximize innate immune responses to pathogens. TLRs actively inhibit a classical ER-stress response by blocking PERK and ATF6 while promoting IRE1 and XBP-1 activation hence co-opting or hijacking the ancestral stress pathway. This intriguing behavior suggests that the non-traditional activation of the IRE1/XBP-1 arm at the expense of the classic ER stress response may be vital to the effective handling of pathogens perhaps by conserving cellular energy resources or by avoiding classic ER-stress-induced apoptosis. In contrast, TLR agonists may initiate or exacerbate inflammatory diseases that are ER-stress related (J. H. Lin, P. Walter, T. S. Yen, Annu Rev Pathol (Oct. 3, 2007)). Diseases such as atherosclerosis, cystic fibrosis, inflammatory bowel disease and type 2 diabetes, display features characteristic of ER-stress, including the production of ER-stress induced chaperones, neutrophil and macrophage infiltration and increased acute phase proteins. Indeed, the recently demonstrated role of XBP-1 in inflammatory bowel disease (A. Kaser et al., Cell 134, 743 (Sep. 5, 2008)) and type 2 diabetes (U. Ozcan et al., Science 306, 457 (Oct. 15, 2004)) raises the possibility that XBP-1 contributes to inflammation in these diseases by affecting TLR signaling. Therefore, ER-stress acts as either an adjuvant to amplify protective TLR mediated responses in the setting of vaccination, or natural infection or may further exacerbate harmful TLR-driven proinflammatory responses.

### Equivalents

Those skilled in the art will recognize, or be able to ascertain using no more than routine experimentation, many equivalents to the specific embodiments and methods described herein. Such equivalents are intended to be encompassed by the scope of the following claims.

What is claimed is:

1. A method of identifying a compound that is useful in increasing Toll like receptor-(TLR) mediated signaling comprising,
a) providing an indicator composition comprising an XBP-1 polypeptide;
b) contacting the indicator composition with each member of a library of compounds;
c) determining the effect of the compound on XBP-1 activity;
d) selecting a compound of interest that increases XBP-1 activity as compared to an appropriate control;
e) determining the effect of the compound on TLR-mediated signaling;
f) selecting a compound of interest that increases TLR-mediated signaling as compared to an appropriate control, thereby identifying the compound as useful in increasing TLR-mediated signaling.

2. A method of identifying a compound that is useful in increasing Toll like receptor-(TLR) mediated signaling comprising,
a) providing an indicator composition comprising an IRE-1 polypeptide;
b) contacting the indicator composition with each member of a library of compounds;
c) determining the effect of the compound on IRE-1 activity;
d) selecting a compound of interest that increases IRE-1 activity as compared to an appropriate control;
e) determining the effect of the compound on TLR-mediated signaling;
f) selecting a compound of interest that increases TLR-mediated signaling as compared to an appropriate control, thereby identifying the compound as useful in increasing TLR-mediated signaling.

3. A method of identifying a compound that is useful in decreasing Toll like receptor-(TLR) mediated signaling comprising,
a) providing an indicator composition comprising an XBP-1 polypeptide;
b) contacting the indicator composition with each member of a library of compounds;
c) determining the effect of the compound on XBP-1 activity;
d) selecting a compound of interest that decreases XBP-1 activity as compared to an appropriate control; and
e) determining the effect of the compound on TLR-mediated signaling;
f) selecting a compound of interest that decreases TLR-mediated signaling as compared to an appropriate control, thereby identifying the compound as useful in decreasing TLR-mediated signaling.

4. A method of identifying a compound that is useful in decreasing like receptor-(TLR) mediated signaling comprising,
a) providing an indicator composition comprising an IRE-1 polypeptide;
b) contacting the indicator composition with each member of a library of compounds;
c) determining the effect of the compound on IRE-1 activity;
d) selecting a compound of interest that decreases IRE-1 activity as compared to an appropriate control;
e) determining the effect of the compound on TLR-mediated signaling;
f) selecting a compound of interest that decreases TLR-mediated signaling as compared to an appropriate control, thereby identifying the compound as useful in increasing TLR-mediated signaling.

5. The method of claim 3, wherein the effect of the compound on TLR-mediated signaling is determined by measuring the effect of the compound on the production of a proinflammatory cytokine.

6. The method of claim 5, wherein the effect of the compound on the production of a proinflammatory cytokine is is determined by measuring the effect of the compound on sustained production of the proinflammatory cytokine.

7. The method of claim 5, wherein the proinflammatory cytokine is IL-6.

8. The method of claim 5, wherein the proinflammatory cytokine is IFN.

9. The method of claim 5, wherein the proinflammatory cytokine is ISG15.

10. The method of claim 3, wherein the activity of XBP-1 is determined by measuring XBP-1 splicing.

11. The method of claim 3, wherein the activity of XBP-1 is determined by assaying XBP-1 protein levels.

12. The method of claim 3, further comprising determining the activation of MyD88, TRIF, TRAF6, or NADPH oxidase.

13. The method of claim 4, wherein the activity of IRE-1 is determined by measuring IRE-1 kinase activity.

14. The method of claim 2, wherein the activity of IRE-1 is determined by measuring IRE-1 endoribonuclease activity.

15. The method of claim 4, wherein the activity of IRE-1 is determined by measuring the binding of IRE-1 to XBP-1.

16. The method of claim 4, wherein the activity of IRE-1 is determined by measuring IRE-1 protein levels.

17. The method of claim 3, wherein the TLR-mediated signaling is TLR2-mediated signaling.

18. The method of claim 3, wherein the TLR-mediated signaling is TLR4-mediated signaling.

19. The method of claim 3, wherein the TLR-mediated signaling is TLR5-mediated signaling.

20. The method of claim 3, wherein the indicator composition is a hematopoietic cell.

21. The method of claim 3, wherein the cell has been engineered to express the XBP-1 polypeptide by introducing into the cell an expression vector encoding the polypeptide.

22. The method of claim 21, wherein the cell is a macrophage.

23. The method of claim 21, wherein the cell is a dendritic cell.

24. The method of claim 21, wherein the cell is under ER stress.

25. The method of claim 21, wherein the cell is contacted with an agent that activates TLR signaling.

26. The method of claim 3, wherein the indicator composition is a cell free composition.

27. The method of claim 3, further comprising determining the effect of the identified compound on PERK and/or ATF6 activity.

28. The method of claim 3, further comprising determining the effect of the compound on XBP-1 mRNA splicing and/or XBP-1 protein production.

29. The method of claim 3, further comprising determining the effect of the compound on the activation of immunoglobulin production.

30. The method of claim 29, further comprising determining the effect of the compound on the activation of LPS-stimulated antibody production.

31. The method of claim 3, further comprising determining the effect of the compound on a response of an immune cell to a bacterial pathogen.

32. The method of claim 3, further comprising determining the effect of the identified test compound on an innate immune response in a non-human animal, comprising administering the test compound to the animal, measuring an effect of TLR-mediated signaling on an innate immune response in the animal in the presence and absence of the test compound, and selecting a compound that modulates TLR-mediated signaling in the animal to thereby determine the effect of the test compound identified on an innate immune response in the animal.

33. The method of claim 32, wherein the non-human animal model is a model of infection with a bacterial pathogen.

34. A method for increasing Toll like receptor signaling in a macrophage, comprising contacting a macrophage with an agent that increases the biological activity of XBP-1 in the macrophage, wherein the agent is selected from the group consisting of: a nucleic acid molecule encoding an XBP-1 polypeptide, a nucleic acid molecule encoding an XBP-1 polypeptide, or combinations thereof, such that Toll like receptor signaling is increased in the macrophage.

35. The method of claim 34, wherein the step of contacting occurs in vitro.

36. The method of claim 34, wherein the step of contacting occurs in vivo.

37. The method of claim 34, wherein TLR-mediated signaling is initiated by an agent that binds to a Toll like receptor selected from the group consisting of Toll like receptor 2, 4, and 5.

38. The method of claim 37, wherein the agent activates MyD88, TRIF, TRAF6, or NADPH oxidase.

39. The method of claim 37, wherein the agent is a pathogen.

40. The method of claim 37, wherein the agent is selected from the group consisting of: lipopolysaccharide (LPS), lipoteichoic acid, PAM3CSK4, or FSL1.

41. The method of claim 34, further comprising assaying for increases TLR-mediated signaling.

42. The method of claim 34, further comprising stimulating ER stress in the cell.


SEQUENCE LISTING (49 sequences)

SEQ ID NO: 1 - DNA, 26 bp, Artificial Sequence
  Synthetic oligonucleotide
cagcactcagactacgtgcacctctg

SEQ ID NO: 2 - DNA, 21 bp, Artificial Sequence
  Synthetic oligonucleotide
gggattcatgaatggccctta

SEQ ID NO: 3 - DNA, 21 bp, Artificial Sequence
  Synthetic siRNA
guuggacccugucauguuutt

SEQ ID NO: 4 - DNA, 21 bp, Artificial Sequence
  Synthetic siRNA
aaacaugacaggguccaactt

SEQ ID NO: 5 - DNA, 21 bp, Artificial Sequence
  Synthetic siRNA
gccauuaaugaacucauuctt

SEQ ID NO: 6 - DNA, 21 bp, Artificial Sequence
  Synthetic siRNA
gaaugaguucauuaauggctt

SEQ ID NO: 7 - RNA, 21 nt, Artificial Sequence
  Synthetic siRNA
gaagagaaccacaaacuccuu

SEQ ID NO: 8 - RNA, 21 nt, Artificial Sequence
  Synthetic siRNA
ggaguuugugguucucuucuu

SEQ ID NO: 9 - RNA, 21 nt, Artificial Sequence
  Synthetic siRNA
gaggaucacccugaauucauu

SEQ ID NO: 10 - RNA, 21 nt, Artificial Sequence
  Synthetic siRNA
ugaauucagggugauccucuu

SEQ ID NO: 11 - RNA, 21 nt, Artificial Sequence
  Synthetic siRNA
cuuggacccagucauguucuu

SEQ ID NO: 12 - RNA, 21 nt, Artificial Sequence
  Synthetic siRNA
gaacaugacuggguccaaguu

SEQ ID NO: 13 - RNA, 21 nt, Artificial Sequence
  Synthetic siRNA
aucugcuuucauccagccauu

SEQ ID NO: 14 - RNA, 21 nt, Artificial Sequence
  Synthetic siRNA
uggcuggaugaaagcagauuu

SEQ ID NO: 15 - RNA, 21 nt, Artificial Sequence
  Synthetic siRNA
gccccuagucuuagagauauu

SEQ ID NO: 16 - RNA, 21 nt, Artificial Sequence
  Synthetic siRNA
uaucucuaagacuaggggcuu

SEQ ID NO: 17 - RNA, 21 nt, Artificial Sequence
  Synthetic siRNA
gaaccuguagaagaugaccuu

SEQ ID NO: 18 - RNA, 21 nt, Artificial Sequence
  Synthetic siRNA
ggucaucuucuacagguucuu

SEQ ID NO: 19 - RNA, 21 nt, Artificial Sequence
  Synthetic siRNA
cguauacaggcugccaucauu

SEQ ID NO: 20 - RNA, 21 nt, Artificial Sequence
  Synthetic siRNA
caagcucaacuacuugagguu

SEQ ID NO: 21 - RNA, 21 nt, Artificial Sequence
  Synthetic siRNA
ccucaaguaguugagcuuguu

SEQ ID NO: 22 - DNA, 15 bp, Artificial Sequence
  Synthetic oligonucleotide
tggatgacgtgtaca

SEQ ID NO: 23 - DNA, 28 bp, Artificial Sequence
  Synthetic oligonucleotide
tcgagacaggtgctgacgtggcgattcc

SEQ ID NO: 24 - RNA, 21 nt, Artificial Sequence
  Synthetic oligonucleotide
ugauggcagccuguauacguu

SEQ ID NO: 25 - DNA, 25 bp, Artificial Sequence
  Synthetic oligonucleotide
caaccaacaagtgatattctccatg

SEQ ID NO: 26 - DNA, 21 bp, Artificial Sequence
  Synthetic oligonucleotide
gatccacactctccagctgca

SEQ ID NO: 27 - DNA, 23 bp, Artificial Sequence
  Synthetic oligonucleotide
gaggataccactcccaacagacc

SEQ ID NO: 28 - DNA, 24 bp, Artificial Sequence
  Synthetic oligonucleotide
aagtgcatcatcgttgttcataca

SEQ ID NO: 29 - DNA, 21 bp, Artificial Sequence
  Synthetic oligonucleotide
agcggaacaagtcacgaagac

SEQ ID NO: 30 - DNA, 20 bp, Artificial Sequence
  Synthetic oligonucleotide
tggggctttaggccatactc

SEQ ID NO: 31 - DNA, 25 bp, Artificial Sequence
  Synthetic oligonucleotide
catcttctcaaaattcgagtgacaa

SEQ ID NO: 32 - DNA, 23 bp, Artificial Sequence
  Synthetic oligonucleotide
tgggagtagacaaggtacaaccc

SEQ ID NO: 33 - DNA, 20 bp, Artificial Sequence
  Synthetic oligonucleotide
taccaccatgtacccaggca

SEQ ID NO: 34 - DNA, 24 bp, Artificial Sequence
  Synthetic oligonucleotide
ctcaggaggagcaatgatcttgat

SEQ ID NO: 35 - DNA, 18 bp, Artificial Sequence
  Synthetic oligonucleotide
tcatcggacgcacttgga

SEQ ID NO: 36 - DNA, 21 bp, Artificial Sequence
  Synthetic oligonucleotide
caaccaccttgaatggcaaga

SEQ ID NO: 37 - DNA, 21 bp, Artificial Sequence
  Synthetic oligonucleotide
gtccctagcttggctgacaga

SEQ ID NO: 38 - DNA, 18 bp, Artificial Sequence
  Synthetic oligonucleotide
tggagagcgagggctttg

SEQ ID NO: 39 - DNA, 21 bp, Artificial Sequence
  Synthetic oligonucleotide
caagatcaagccccacctgat

SEQ ID NO: 40 - DNA, 21 bp, Artificial Sequence
  Synthetic oligonucleotide
agttcgccccaaccagtactt

SEQ ID NO: 41 - DNA, 22 bp, Artificial Sequence
  Synthetic oligonucleotide
ccccagtgtcaaactgtaccag

SEQ ID NO: 42 - DNA, 24 bp, Artificial Sequence
  Synthetic oligonucleotide
agcgtttccaattttccataaatt

SEQ ID NO: 43 - DNA, 20 bp, Artificial Sequence
  Synthetic oligonucleotide
ccatcaacatgctcccgttc

SEQ ID NO: 44 - DNA, 16 bp, Artificial Sequence
  Synthetic oligonucleotide
gggtaggcctcgccat

SEQ ID NO: 45 - DNA, 20 bp, Artificial Sequence
  Synthetic oligonucleotide
acacgcttgggaatggacac

SEQ ID NO: 46 - DNA, 20 bp, Artificial Sequence
  Synthetic oligonucleotide
ccatgggaagatgttctggg

SEQ ID NO: 47 - DNA, 21 bp, Artificial Sequence
  Synthetic oligonucleotide
gctcgtgaattgatagagaaa

SEQ ID NO: 48 - DNA, 21 bp, Artificial Sequence
  Synthetic oligonucleotide
cccaggctgttcataatgtta

SEQ ID NO: 49 - DNA, 21 bp, Artificial Sequence
  Synthetic oligonucleotide
agactacgacagccacattaa